(19) United States
(12) Patent Application Publication
(10) Pub. No.: US 2026/0174117 A1
(43) Pub. Date: Jun. 25, 2026

(54) COMPOSITIONS AND METHODS BASED ON QPT ENGINEERING FOR PRODUCING TOBACCO PLANTS AND PRODUCTS HAVING ALTERED ALKALOID LEVELS

(71) Applicant: Altria Client Services LLC, Richmond, VA (US)

(72) Inventors: Dong QI, Henrico, VA (US); Chengalrayan KUDITHIPUDI, Midlothian, VA (US); Yanxin SHEN, Henrico, VA (US)

(73) Assignee: Altria Client Services LLC, Richmond, VA (US)

(21) Appl. No.: 19/556,630

(22) Filed: Mar. 4, 2026

Related U.S. Application Data
(63) Continuation of application No. 17/767,692, now Pat. No. 12,590,315.
(60) Provisional application No. 62/913,357, filed on Oct. 10, 2019.

(51) Int. Cl. C12N 15/82; A24B 13/00; A24B 15/12

(52) U.S. Cl. CPC A24B 13/00; A24B 15/12; C12N 15/8243

(57) ABSTRACT

The present disclosure provides compositions and methods related to tobacco plants with altered total alkaloid and nicotine levels and commercially acceptable leaf grade, their development via breeding or transgenic approaches, and production of tobacco products from these tobacco plants.

### CROSS-REFERENCE TO RELATED APPLICATION

This application is a continuation of U.S. patent application Ser. No. 17/767,692, filed Apr. 8, 2022, which is a U.S. National Phase application of International Patent Application No. PCT/US2020/055105, filed Oct. 9, 2020, which in turn claims the benefit of U.S. Provisional Application No. 62/913,357, filed Oct. 10, 2019, which are incorporated by reference in their entirety herein.

### INCORPORATION OF SEQUENCE LISTING

A sequence listing contained in the file named “P34736US01_SL.xml” which is 51,322 bytes (measured in operating system MS-Windows®), produced on Mar. 2, 2026, containing a total number of 22 sequences, starting from SEQ ID NO: 1 to SEQ ID NO: 22, is filed electronically herewith and incorporated by reference in its entirety.

### FIELD

The present disclosure provides tobacco genetic engineering for modulating alkaloid and nicotine levels.

### BACKGROUND

Nicotine is the predominant alkaloid, usually accounting for more than 90-95% of the total alkaloids in commercial tobacco cultivars. The remaining alkaloid fraction is primarily comprised three additional alkaloids: nornicotine, anabasine, and anatabine. Tobacco plants with reduced nicotine levels have been achieved with varying and inconsistent results by modulating different nicotine biosynthetic genes and transcriptional regulators. There is a need for new technologies to reduce nicotine levels in tobacco leaves.

### SUMMARY

The present disclosure provides tobacco plants with altered total alkaloid and nicotine levels and commercially acceptable leaf grade, their development via breeding or transgenic approaches, and production of tobacco products from these tobacco plants.

In an aspect, the present disclosure provides a tobacco plant, or part thereof, comprising one or more mutant alleles in at least one OPT gene selected from the group consisting of QPT1a (g98654), QPT1b (g82210), QPT2a (g82211), and QPT2b (g98655), wherein the tobacco plant is capable of producing a leaf comprising a nicotine level less than the nicotine level of a leaf from a control tobacco plant not having the one or more mutant alleles when grown and processed under comparable conditions.

In another aspect, a tobacco plant comprises one or more mutant alleles in at least two QPT genes selected from the group consisting of OPT1a, QPT1b, QPT2a, and QPT2b.

In a further aspect, a tobacco plant comprises one or more mutant alleles in at least three OPT genes selected from the group consisting of OPT1a, QPT1b, QPT2a, and QPT2b.

In another aspect, a tobacco plant comprises one or more mutant alleles in at least four OPT genes selected from the group consisting of OPT1a, OPT1b, QPT2a, and QPT2b.

In an aspect, the present disclosure provides a tobacco plant selected from the group consisting of a single qpt mutant, a double qpt mutant, a triple qpt mutant, and a quadruple qpt mutant.

In an aspect, the present disclosure provides a tobacco plant as listed in Tables 11A and 11B. In another aspect, the present disclosure provides a progeny plant of a tobacco plant in Tables 11A and 11B, from either selfing or a cross with another plant in Tables 11A and 11B.

In another aspect, the present disclosure provides a tobacco plant comprising various combinations of the qpt mutant alleles listed in Tables 11A and 11B to give rise to a single qpt mutant or a double qpt mutant.

The present disclosure further provides cured tobacco, tobacco blends, tobacco products comprising plant material from tobacco plants, lines, varieties or hybrids disclosed.

### BRIEF DESCRIPTION OF THE SEQUENCES

SEQ ID NOs: 1 to 4 set forth exemplary genomic sequences of OPT1a, QPT1b, QPT2a, and QPT2b, respectively, from a TN90 reference genome.

SEQ ID NOs: 5 to 8 set forth exemplary cDNA sequences of OPT1a, QPT1b, QPT2a, and QPT2b, respectively, from TN90.

SEQ ID NOs: 9 to 12 set forth exemplary polypeptide sequences of OPT1a, QPT1b, QPT2a, and QPT2b, respectively, from TN90.

SEQ ID NO: 13 sets forth an example guide RNA sequence.

### BRIEF DESCRIPTION OF THE DRAWINGS

FIG. 1: Genetic map of OPT genes in the tobacco genome.

FIG. 2: RNA expression of four OPT genes in TN90 roots. RPKM=reads per kilobase of transcript per million mapped reads.

FIG. 3: Depiction of candidate tobacco calli comprising knock-out indels as identified by fragment analysis. Upper panels show the fragment analysis result of both QPT2a and QPT2b from the TN90 candidate callus #44. The lower panel shows the fragment analysis result of QPT2b from the K326 candidate callus #139. The unmodified QPT gene fragments are shaded, and the edited QPT gene fragments are shaded and denoted with a black star.

### DETAILED DESCRIPTION

Unless defined otherwise, technical and scientific terms used herein have the same meaning as commonly understood by one of ordinary skill in the art. One skilled in the art will recognize many methods can be used in the practice of the present disclosure. Indeed, the present disclosure is in no way limited to the methods and materials described. For purposes of the present disclosure, the following terms are defined below.

Any references cited herein, including, e.g., all patents and publications are incorporated by reference in their entirety.

As used herein, the singular form “a,” “an,’ and “the” include plural references unless the context clearly dictates otherwise. For example, the term “a compound” or “at least one compound” may include a plurality of compounds, including mixtures thereof.

When the term “about” is used in conjunction with a numerical range, it modifies that range by extending the boundaries above and below the numerical values set forth by 10%.

As used herein, phrases such as “less than”, “more than”, “at least”, “at most”, “approximately”, “below”, “above”, and “about”, when used in conjunction with a series of numerical values, modify each and every value within the series. For example, an expression of “less than 1%, 2%, or 3%” is equivalent to “less than 1%, less than 2%, or less than 3%.”

As used herein, a tobacco plant refers to a plant from the species Nicotiana tabacum.

Nicotine biosynthesis in tobacco involves the conversion of aspartate to α-iminosuccinate by the enzymes ASPARTATE OXIDASE1 (AO1) and (AO2). From there, α-iminosuccinate is converted to quinolinate by QUINOLINATE SYNTHASE (QS). Quinolate is then converted to nicotianate mononucleotide by QUINOLINATE PHOSPHORIBOSYL TRANSFERASE1 (QPT1) and QPT2. Nicotianate mononucleotide can then be used by tobacco as a direct precursor of nicotine.

QPT enzymes are classified under the enzyme classification system as EC 2.4.2.19. In Nicotiana tabacum, four genes encode quinolinate phosphoribosyl transferases, designated QPT1a, QPT1b, QPT2a, and QPT2b. Table 8A lists genomic DNA sequences, cDNA sequences, and protein sequences of these four OPT genes in a TN90 plant. The present disclosure describes compositions and methods that are used to edit QPT genes to produce qpt mutant plants having reduced nicotine levels while maintaining leaf quality.

As used herein, “QPT1a” or the “OPT1a gene” refers to a genic locus in tobacco encoding a polypeptide having an exemplary amino acid sequence in TN90 as set forth in SEQ ID No. 9.

As used herein, “QPT1b” or the “OPT1b gene” refers to a genic locus in tobacco encoding a polypeptide having an exemplary amino acid sequence in TN90 as set forth in SEQ ID No. 10.

As used herein, “QPT2a” or the “QPT2a gene” refers to a genic locus in tobacco encoding a polypeptide having an exemplary amino acid sequence in TN90 as set forth in SEQ ID No. 11.

As used herein, “QPT2b” or the “QPT2b gene” refers to a genic locus in tobacco encoding a polypeptide having an exemplary amino acid sequence in TN90 as set forth in SEQ ID No. 12.

As used herein, a mutation refers to an inheritable genetic modification introduced into a gene to reduce, inhibit, or eliminate the expression or activity of a product encoded by the gene. Such a modification can be in any sequence region of a gene, for example, in a promoter, 5′ UTR, exon, intron, 3′ UTR, or terminator region. In an aspect, mutations are not natural polymorphisms that exist in a particular tobacco variety or cultivar. As used herein, a “mutant allele” refers to an allele from a locus where the allele comprises a mutation.

As used herein, a “qpt mutant” refers to a tobacco plant comprising one or more mutations in one or more QPT genes. A qpt mutant can be a single mutant, a double mutant, a triple mutant, or a quadruple mutant. As used herein, a single, double, triple, or quadruple qpt mutant refers to a mutant having modifications in one, two, three, or four QPT genes, respectively. A qpt mutant can also be a homozygous mutant, a heterozygous mutant, or a heteroallelic mutant combination in one or more QPT genes.

As used herein, a gene name or a genic locus name is capitalized and shown in italic, e.g., OPT1a, OPT1b, QPT2a, and QPT2b. A protein or polypeptide name is capitalized without being italicized, e.g., QPT1a, QPT1b, QPT2a, and QPT2b. A mutant name (for either referencing to a general mutation in a gene or a group of genes, or referencing to a specific mutant allele) is shown in lower case and italic, e.g., qpt, qpt1a, qpt1b, qpt2a, and qpt2b.

In an aspect, the present disclosure provides a tobacco plant, or part thereof, comprising one or more mutant alleles in at least one OPT gene selected from the group consisting of OPT1a, QPT1b, QPT2a, and QPT2b, wherein the tobacco plant is capable of producing a leaf comprising a nicotine level less than the nicotine level of a leaf from a control tobacco plant not having the one or more mutant alleles when grown and processed under comparable conditions. In an aspect, a single qpt mutant tobacco plant is provided. In another aspect, a single qpt mutant tobacco plant comprises nicotine at a level below 1%, below 2%, below 5%, below 8%, below 10%, below 12%, below 15%, below 20%, below 25%, below 30%, below 40%, below 50%, below 60%, below 70%, below 80%, below 90%, or below 95% of the nicotine level of a control plant not having the single qpt mutation when grown in similar growth conditions. In a further aspect, a single qpt mutant tobacco plant comprises nicotine at a level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control plant not having the single qpt mutation when grown in similar growth conditions.

In another aspect, a tobacco plant comprises one or more mutant alleles in at least two QPT genes selected from the group consisting of OPT1a, QPT1b, QPT2a, and QPT2b. In an aspect, a double qpt mutant tobacco plant is provided. In another aspect, a double qpt mutant tobacco plant comprises nicotine at a level below 1%, below 2%, below 5%, below 8%, below 10%, below 12%, below 15%, below 20%, below 25%, below 30%, below 40%, below 50%, below 60%, below 70%, below 80%, below 90%, or below 95% of the nicotine level of a control plant not having the double qpt mutations when grown in similar growth conditions. In a further aspect, a double qpt mutant tobacco plant comprises nicotine at a level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control plant not having the double qpt mutations when grown in similar growth conditions.

In a further aspect, a tobacco plant comprises one or more mutant alleles in at least three OPT genes selected from the group consisting of OPT1a, OPT1b, QPT2a, and QPT2b. In an aspect, a triple qpt mutant tobacco plant is provided. In another aspect, a triple qpt mutant tobacco plant comprises nicotine at a level below 1%, below 2%, below 5%, below 8%, below 10%, below 12%, below 15%, below 20%, below 25%, below 30%, below 40%, below 50%, below 60%, below 70%, below 80%, below 90%, or below 95% of the nicotine level of a control plant not having the triple qpt mutations when grown in similar growth conditions. In a further aspect, a triple qpt mutant tobacco plant comprises nicotine at a level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control plant not having the triple qpt mutations when grown in similar growth conditions.

In another aspect, a tobacco plant comprises one or more mutant alleles in at least four QPT genes selected from the group consisting of OPT1a, QPT1b, QPT2a, and QPT2b. In an aspect, a quadruple qpt mutant tobacco plant is provided. In another aspect, a quadruple qpt mutant tobacco plant comprises nicotine at a level below 1%, below 2%, below 5%, below 8%, below 10%, below 12%, below 15%, below 20%, below 25%, below 30%, below 40%, below 50%, below 60%, below 70%, below 80%, below 90%, or below 95% of the nicotine level of a control plant not having the quadruple qpt mutations when grown in similar growth conditions. In a further aspect, a quadruple qpt mutant tobacco plant comprises nicotine at a level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control plant not having the quadruple qpt mutations when grown in similar growth conditions.

In an aspect, a tobacco plant provided herein is a single qpt mutant, a double qpt mutant, a triple qpt mutant, or a quadruple qpt mutant. In another aspect, a tobacco plant comprises one or more qpt mutant alleles listed in Tables 11A and 11B. Each and every combination of the qpt mutant alleles listed in Tables 11A and 11B is also provided to give rise to a single qpt mutant, a double qpt mutant, a triple qpt mutant, or a quadruple qpt mutant. Each of the mutated loci can be either homozygous or heterozygous, or comprises a heteroallelic combination. In another aspect, a tobacco plant comprises a qpt mutant genotype combination as shown for each individual line listed in Tables 11A and 11B.

In an aspect, a tobacco plant is capable of producing a leaf comprising a nicotine level less than 90%, 80%, 70%, 60%, 50%, 40%, 30%, 20%, 15%, 10%, 9%, 8%, 7%, 6%, 5%, 4%, 3%, 2%, 1%, 0.5%, or 0.25% of the nicotine level of a leaf from a control tobacco plant when grown and processed under comparable conditions. In another aspect, a tobacco plant is capable of producing a leaf comprising a nicotine level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control tobacco plant when grown and processed under comparable conditions.

In another aspect, a tobacco plant is capable of producing a leaf comprising a total alkaloid level less than 90%, 80%, 70%, 60%, 50%, 40%, 30%, 20%, 15%, 10%, 9%, 8%, 7%, 6%, 5%, 4%, 3%, 2%, 1%, 0.5%, or 0.25% of the total alkaloid level of a leaf from a control tobacco plant when grown and processed under comparable conditions. In another aspect, a tobacco plant is capable of producing a leaf comprising a total alkaloid level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the total alkaloid level of a control tobacco plant when grown and processed under comparable conditions.

In a further aspect, a tobacco plant is capable of producing a leaf comprising a total alkaloid level less than 40%, 35%, 30%, 25%, 20%, 15%, 10%, or 5% of the total alkaloid level of a leaf from a control tobacco plant when grown and processed under comparable conditions.

In an aspect, a mutant qpt allele comprises a mutation in a OPT sequence region selected from the group consisting of a promoter, 5′ UTR, first exon, first intron, second exon, second intron, third exon, third intron, fourth exon, fourth intron, fifth exon, fifth intron, sixth exon, sixth intron, seventh exon, seventh intron, eighth exon, 3′ UTR, terminator, and any combination thereof. In another aspect, a mutant qpt allele comprises a mutation in a OPT genomic sequence region listed in Tables 8D, 8E, 8F, and 8G.

In another aspect, a mutant qpt allele comprises one or more mutation types selected from the group consisting of a nonsense mutation, a missense mutation, a frameshift mutation, a splice-site mutation, and any combination thereof. In an aspect, a mutant qpt allele is a null allele or a knock-out allele.

In an aspect, a mutant qpt allele results in one or more of the following: a QPT protein truncation, a non-translatable QPT gene transcript, a non-functional QPT protein, a premature stop codon in a OPT gene, and any combination thereof.

In another aspect, a mutant qpt allele comprises a mutation selected from the group consisting of a substitution, a deletion, an insertion, a duplication, and an inversion of one or more nucleotides relative to a wild-type OPT gene.

In an aspect, a qpt mutant comprises a zygosity status selected from the group consisting of homozygous, heterozygous, and heteroallelic. In another aspect, a qpt mutant is homozygous or heteroallelic in at least one, two, three, or four QPT genes. In an aspect, a qpt mutant is homozygous or heteroallelic in at least one QPT gene. In an aspect, a qpt mutant is homozygous or heteroallelic in at least two QPT genes. In an aspect, a qpt mutant is homozygous or heteroallelic in at least three QPT genes. In an aspect, a qpt mutant is homozygous or heteroallelic in at least four QPT genes.

In an aspect, a qpt mutant comprises at least one mutation in QPT1a. In another aspect, a qpt mutant comprises at least one mutation in QPT1b. In another aspect, a qpt mutant comprises at least one mutation in QPT2a. In another aspect, a qpt mutant comprises at least one mutation in QPT2b.

In an aspect, a qpt mutant comprises mutations in QPT2a and QPT2b. In another aspect, a qpt mutant comprises mutations in QPT1a and QPT1b. In another aspect, a qpt mutant comprises mutations in QPT1a and QPT2a. In another aspect, a qpt mutant comprises mutations in QPT1a and QPT2b. In another aspect, a qpt mutant comprises mutations in QPT2a and QPT1b. In another aspect, a qpt mutant comprises mutations in OPT1b and QPT2b.

In an aspect, a qpt mutant comprises mutations in OPT1a, OPT1b, and QPT2a. In another aspect, a qpt mutant comprises mutations in QPT1a, OPT1b, and QPT2b. In another aspect, a qpt mutant comprises mutations in OPT1a, QPT2a, and QPT2b. In another aspect, a qpt mutant comprises mutations in QPT1b, QPT2a, and QPT2b.

In another aspect, a qpt mutant comprises mutations in QPT1a, OPT1b, QPT2a, and QPT2b.

In an aspect, a tobacco plant is capable of producing a leaf comprising a nicotine level selected from the group consisting of less than 0.15%, less than 0.125%, less than 0.1%, less than 0.08%, less than 0.06%, less than 0.05%, less than 0.04%, less than 0.03%, less than 0.02%, and less than 0.01% dry weight.

In another aspect, a tobacco plant is capable of producing a leaf comprising a total alkaloid level selected from the group consisting of less than 1%, less than 0.8%, less than 0.7%, less than 0.6%, less than 0.5%, less than 0.4%, less than 0.3%, and less than 0.2% dry weight.

In a further aspect, a tobacco plant is capable of producing a cured leaf comprising a total TSNA level of between 2 and 0.05, between 1.9 and 0.05, between 1.8 and 0.05, between 1.7 and 0.05, between 1.6 and 0.05, between 1.5 and 0.05, between 1.4 and 0.05, between 1.3 and 0.05, between 1.2 and 0.05, between 1.1 and 0.05, between 1.0 and 0.05, between 0.9 and 0.05, between 0.8 and 0.05, between 0.7 and 0.05, between 0.6 and 0.05, between 0.5 and 0.05, between 0.4 and 0.05, between 0.3 and 0.05, between 0.2 and 0.05, between 0.15 and 0.05, or between 0.1 and 0.05 parts per million (ppm).

In an aspect, a tobacco plant is capable of producing leaves, when cured, having a USDA grade index value selected from the group consisting of 50 or more, 55 or more, 60 or more, 65 or more, 70 or more, 75 or more, 80 or more, 85 or more, 90 or more, and 95 or more. In another aspect, a tobacco plant is capable of producing leaves, when cured, having a USDA grade index value comparable to that of a control plant when grown and cured in similar conditions, where the control plant shares an essentially identical genetic background with the tobacco plant except for the modification. In a further aspect, a tobacco plant is capable of producing leaves, when cured, having a USDA grade index value of at least about 65%, at least about 70%, at least about 75%, at least about 80%, at least about 85%, at least about 90%, at least about 95%, or at least about 98% of the USDA grade index value of a control plant when grown in similar conditions, where the control plant shares an essentially identical genetic background with the tobacco plant except the modification. In a further aspect, a tobacco plant is capable of producing leaves, when cured, having a USDA grade index value of between 65% and 130%, between 70% and 130%, between 75% and 130%, between 80% and 130%, between 85% and 130%, between 90% and 130%, between 95% and 130%, between 100% and 130%, between 105% and 130%, between 110% and 130%, between 115% and 130%, or between 120% and 130% of the USDA grade index value of a control plant. In a further aspect, a tobacco plant is capable of producing leaves, when cured, having a USDA grade index value of between 70% and 125%, between 75% and 120%, between 80% and 115%, between 85% and 110%, or between 90% and 100% of the USDA grade index value of a control plant.

In an aspect, a tobacco plant comprises nicotine at a level below 1%, below 2%, below 5%, below 8%, below 10%, below 12%, below 15%, below 20%, below 25%, below 30%, below 40%, below 50%, below 60%, below 70%, or below 80% of the nicotine level of a control plant when grown in similar growth conditions, where the control plant shares an essentially identical genetic background with the tobacco plant except for the modification.

In a further aspect, a tobacco plant comprises one or more qpt mutant alleles and further comprises a transgene or mutation directly suppressing the expression or activity of one or more genes encoding a product selected from the group consisting of N-methylputrescine oxidase (MPO), putrescine N-methyltransferase (PMT), berberine bridge enzyme-like (BBL), A622, aspartate oxidase (AO), agmatine deiminase (AIC), arginase, diamine oxidase (DAO), ornithine decarboxylase (ODC), arginine decarboxylase, nicotine uptake permease (NUP), and MATE transporter.

In an aspect, a tobacco plant comprises one or more qpt mutant alleles and further comprises a mutation in an ERF gene of Nic2 locus. In an aspect, a tobacco plant further comprises one or more mutations in two or more, three or more, four or more, five or more, six or more, or all seven genes selected from the group consisting of ERF189, ERF115, ERF221, ERF104, ERF179, ERF17, and ERF168. See Shoji et al., Plant Cell, (10): 3390-409 (2010); and Kajikawa et al.,, Plant physiol. 2017, 174:999-1011. In an aspect, a tobacco plant further comprises one or more mutations in ERF189, ERF115, or both.

In an aspect, a tobacco plant comprises one or more qpt mutant alleles and further comprises a mutation in an ERF gene of Nic1 locus (or Nic1b locus as in PCT/US2019/013345 filed on Jan. 11, 2019, published as WO/2019/140297). See also WO/2018/237107. In an aspect, a tobacco plant further comprises one or more mutations in two or more, three or more, four or more, five or more, six or more, or seven or more genes selected from the group consisting of ERF101, ERF110, ERFnew, ERF199, ERF19, ERF130, ERF16, ERF29, ERF210, and ERF91L2. See Kajikawa et al.,, Plant physiol. 2017, 174:999-1011. In an aspect, a tobacco plant further comprises one or more mutations in one or more, two or more, three or more, four or more, five or more, or all six genes selected from the group consisting of ERFnew, ERF199, ERF19, ERF29, ERF210, and ERF91L2.

A variety of factors affect tobacco alkaloid levels including genotype, environment, fertilization, and agronomic practices (for example, nicotine production is stimulated by topping, wounding, and herbivore damage). Low-alkaloid traits initially found in strains of Cuban cigar tobacco varieties were introduced into cigarette varieties through a series of backcrosses. Low-alkaloid tobacco germplasm was subsequently registered in the genetic background of cultivar Burley 21 (Legg et al., Crop Science, 10:212 (1970)). Genetic studies using the low alkaloid Burley 21 (LA BU21) lines indicated that two unlinked loci contribute to nicotine levels in the tobacco leaf. These two loci are referred to as Nic1 and Nic2. nic1 and nic2 (same as nicotine 1 and nicotine 2, respectively) mutations in LA BU21 are semidominant. They show dose-dependent effects on nicotine levels, with the effects of nic1 about 2.4 times stronger than those of nic2. Molecular characterization of Nic2 locus has been reported. The nic2 mutation was shown to contain a deletion of a cluster of transcription factor genes from the ethylene responsive factor (ERF) family, e.g., ERF189, ERF115, ERF221, ERF104, ERF179, ERF17, and ERF168 (Shoji et al., Plant Cell, (10): 3390-409 (2010)).

Reducing total alkaloid content in tobacco can have many benefits. It can increase the value of tobacco as a biomass resource. Increases in nicotinic alkaloid in tobacco plants may play an important role in protecting plants against insects and herbivores.

Consistent with alkaloids' role in insect defense, LA BU21 was reported to be extremely susceptible to insect damage (Legg et al., Crop Science, 10:212 (1970)). A further study comparing isogenic lines of flue-cured tobacco with low total alkaloids percentage (approximately 0.20%) with their “normal” recurring parents (total alkaloids 1.85 to 2.70%) reported that yield, grade index, total N, and reducing sugar content in the low alkaloid lines were lower than in the normal flue-cured cultivars (Chaplin and Weeks, Crop Science, 16 (3): 416-18 (1976)).

As used herein, a “low alkaloid variety” of tobacco refers to tobacco variety comprising one or more genetic modifications reducing the total alkaloids (measured via dry weight) to a level less than 25% of the total alkaloid level in a control tobacco variety of a substantially similar genetic background except for the one or more genetic modifications. As a non-limiting example, KY171 can serve as a control for the low-alkaloid variety LA KY171.

In an aspect, the present disclosure further provides a qpt mutant tobacco plant, or part thereof, comprising a nicotine or total alkaloid level selected from the group consisting of less than 3%, less than 2.75%, less than 2.5%, less than 2.25%, less than 2.0%, less than 1.75%, less than 1.5%, less than 1.25%, less than 1%, less than 0.9%, less than 0.8%, less than 0.7%, less than 0.6%, less than 0.5%, less than 0.4%, less than 0.3%, less than 0.2%, less than 0.1%, less than 0.05%, less than 0.025%, less than 0.01%, and less than 0.005%, where the tobacco plant is capable of producing leaves, when cured, having a USDA grade index value of 50 or more 55 or more, 60 or more, 65 or more, 70 or more, 75 or more, 80 or more, 85 or more, 90 or more, and 95 or more. In another aspect, such qpt mutant tobacco plant comprises a nicotine level of less than 0.02% and are capable of producing leaves, when cured, having a USDA grade index value of 70 or more. In a further aspect, such tobacco plant comprises a nicotine level of less than 0.01% and are capable of producing leaves, when cured, having a USDA grade index value of 70 or more.

In an aspect, the present disclosure also provides a tobacco plant, or part thereof, comprising a non-transgenic mutation, where the non-transgenic mutation reduces the nicotine or total alkaloid level of the tobacco plant to below 1%, below 2%, below 5%, below 8%, below 10%, below 12%, below 15%, below 20%, below 25%, below 30%, below 40%, below 50%, below 60%, below 70%, or below 80% of the nicotine level of a control plant when grown in similar growth conditions, where the tobacco plant is capable of producing leaves, when cured, having a USDA grade index value comparable to the USDA grade index value of the control plant, and where the control plant shares an essentially identical genetic background with the tobacco plant except the non-transgenic mutation.

In an aspect, a tobacco plant comprises a qpt mutation introduced by an approach selected from the group consisting of random mutagenesis and targeted mutagenesis. In another aspect, a qpt mutation is introduced by a targeted mutagenesis approach selected from the group consisting of meganuclease, zinc finger nuclease, TALEN, and CRISPR.

Unless specified otherwise, measurements of alkaloid or nicotine levels (or another leaf chemistry or property characterization) or leaf grade index values mentioned herein for a tobacco plant, variety, cultivar, or line refer to average measurements, including, for example, an average of multiple leaves of a single plant or an average measurement from a population of tobacco plants from a single variety, cultivar, or line.

Unless specified otherwise, the nicotine or alkaloid level (or another leaf chemistry or property characterization) of a tobacco plant is measured after topping in a pooled leaf sample collected from leaf number 3, 4, and 5 after topping. As used herein, whenever a comparison between leaves from two plants (e.g., a mutant plant versus a control plant) is mentioned, leaves from the same or comparable stalk position(s) and developmental stage(s) are intended so that the comparison can demonstrate effects due to genotype differences, not from other factors. As an illustration, leaf 3 of a wild-type control plant is intended as a reference point for comparing with leaf 3 of a qpt mutant plant. In an aspect, a tobacco plant comprising at least one qpt mutation is compared to a control tobacco plant of the same tobacco variety.

Nicotine or alkaloid level (or another leaf chemistry or property characterization) of a tobacco plant can also be measured in alternative ways. In an aspect, the nicotine or alkaloid level (or another leaf chemistry or property characterization) of a tobacco plant is measured after topping in a leaf having the highest level of nicotine or alkaloid (or another leaf chemistry or property characterization). In an aspect, the nicotine or alkaloid level of a tobacco plant is measured after topping in leaf number 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In another aspect, the nicotine or alkaloid level (or another leaf chemistry or property characterization) of a tobacco plant is measured after topping in a pool of two or more leaves with consecutive leaf numbers selected from the group consisting of leaf number 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30. In another aspect, the nicotine or alkaloid level (or another leaf chemistry or property characterization) of a tobacco plant is measured after topping in a leaf with a leaf number selected from the group consisting of between 1 and 5, between 6 and 10, between 11 and 15, between 16 and 20, between 21 and 25, and between 26 and 30. In another aspect, the nicotine or alkaloid level (or another leaf chemistry or property characterization) of a tobacco plant is measured after topping in a pool of two or more leaves with leaf numbers selected from the group consisting of between 1 and 5, between 6 and 10, between 11 and 15, between 16 and 20, between 21 and 25, and between 26 and 30. In another aspect, the nicotine or alkaloid level (or another leaf chemistry or property characterization) of a tobacco plant is measured after topping in a pool of three or more leaves with leaf numbers selected from the group consisting of between 1 and 5, between 6 and 10, between 11 and 15, between 16 and 20, between 21 and 25, and between 26 and 30.

As used herein, leaf numbering is based on the leaf position on a tobacco stalk with leaf number 1 being the youngest leaf (at the top) after topping and the highest leaf number assigned to the oldest leaf (at the bottom).

A population of tobacco plants or a collection of tobacco leaves for determining an average measurement (e.g., alkaloid or nicotine level or leaf grading) can be of any size, for example, 5, 10, 15, 20, 25, 30, 35, 40, or 50. Industry-accepted standard protocols are followed for determining average measurements or grade index values.

As used herein, “topping” refers to the removal of the stalk apex, including the shoot apical meristem, flowers, and up to several adjacent leaves, when a tobacco plant is near vegetative maturity and around the start of reproductive growth. Typically, tobacco plants are topped in the button stage (soon after the flower begins to appear). For example, greenhouse or field-grown tobacco plants can be topped when 50% of the plants have at least one open flower. Topping a tobacco plant results in the loss of apical dominance and also induces increased alkaloid production.

Unless indicated otherwise, the nicotine or alkaloid level (or another leaf chemistry or property characterization) of a tobacco plant is measured two weeks after topping. Alternatively, other time points can be used. In an aspect, the nicotine or alkaloid level (or another leaf chemistry or property characterization) of a tobacco plant is measured about 1, 2, 3, 4, or 5 weeks after topping. In another aspect, the nicotine, alkaloid, or polyamine level (or another leaf chemistry or property characterization) of a tobacco plant is measured about 3, 5, 7, 10, 12, 14, 17, 19 or 21 days after topping.

As used herein, “similar growth conditions” or “comparable growth conditions” refer to similar environmental conditions and/or agronomic practices for growing and making meaningful comparisons between two or more plant genotypes so that neither environmental conditions nor agronomic practices would contribute to or explain any difference observed between the two or more plant genotypes. Environmental conditions include, for example, light, temperature, water (humidity), and nutrition (e.g., nitrogen and phosphorus). Agronomic practices include, for example, seeding, clipping, undercutting, transplanting, topping, and suckering. See Chapters 4B and 4C of Tobacco, Production, Chemistry and Technology, Davis & Nielsen, eds., Blackwell Publishing, Oxford (1999), pp 70-103.

“Alkaloids” are complex, nitrogen-containing compounds that naturally occur in plants, and have pharmacological effects in humans and animals. “Nicotine” is the primary natural alkaloid in commercialized cigarette tobacco and accounts for about 90 percent of the alkaloid content in Nicotiana tabacum. Other major alkaloids in tobacco include cotinine, nornicotine, myosmine, nicotyrine, anabasine and anatabine. Minor tobacco alkaloids include nicotine-n-oxide, N-methyl anatabine, N-methyl anabasine, pseudooxynicotine, 2,3 dipyridyl and others.

Alkaloid levels can be assayed by methods known in the art, for example by quantification based on gas-liquid chromatography, high performance liquid chromatography, radio-immunoassays, and enzyme-linked immunosorbent assays. For example, nicotinic alkaloid levels can be measured by a GC-FID method based on CORESTA Recommended Method No. 7, 1987 and ISO Standards (ISO TC 126N 394 E. See also Hibi et al., Plant Physiology 100:826-35 (1992) for a method using gas-liquid chromatography equipped with a capillary column and an FID detector.

Unless specifically indicated otherwise, alkaloids and nicotine levels are measured using a method in accordance with CORESTA Method No 62, Determination of Nicotine in Tobacco and Tobacco Products by Gas Chromatographic Analysis, February 2005, and those defined in the Centers for Disease Control and Prevention's Protocol for Analysis of Nicotine, Total Moisture and pH in Smokeless Tobacco Products, as published in the Federal Register Vol. 64, No. 55 Mar. 23, 1999 (and as amended in Vol. 74, No. 4, Jan. 7, 2009). Alternatively, tobacco total alkaloids can be measured using a segmented-flow colorimetric method developed for analysis of tobacco samples as adapted by Skalar Instrument Co (West Chester, PA) and described by Collins et al., Tobacco Science 13:79-81 (1969). In short, samples of tobacco can be dried, ground, and extracted prior to analysis of total alkaloids and reducing sugars. The method then employs an acetic acid/methanol/water extraction and charcoal for decolorization. Determination of total alkaloids is based on the reaction of cyanogen chloride with nicotine alkaloids in the presence of an aromatic amine to form a colored complex which is measured at 460 nm. Unless specified otherwise, total alkaloid levels or nicotine levels shown herein are on a dry weight basis (e.g., percent total alkaloid or percent nicotine).

In an aspect, a tobacco plant comprises an average nicotine or total alkaloid level selected from the group consisting of about 0.01%, 0.02%, 0.05%, 0.75%, 0.1%, 0.15%, 0.2%, 0.3%, 0.35%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, and 9% on a dry weight basis. In another aspect, a tobacco plant comprises an average nicotine or total alkaloid level selected from the group consisting of about between 0.01% and 0.02%, between 0.02% and 0.05%, between 0.05% and 0.75%, between 0.75% and 0.1%, between 0.1% and 0.15%, between 0.15% and 0.2%, between 0.2% and 0.3%, between 0.3% and 0.35%, between 0.35% and 0.4%, between 0.4% and 0.5%, between 0.5% and 0.6%, between 0.6% and 0.7%, between 0.7% and 0.8%, between 0.8% and 0.9%, between 0.9% and 1%, between 1% and 1.1%, between 1.1% and 1.2%, between 1.2% and 1.3%, between 1.3% and 1.4%, between 1.4% and 1.5%, between 1.5% and 1.6%, between 1.6% and 1.7%, between 1.7% and 1.8%, between 1.8% and 1.9%, between 1.9% and 2%, between 2% and 2.1%, between 2.1% and 2.2%, between 2.2% and 2.3%, between 2.3% and 2.4%, between 2.4% and 2.5%, between 2.5% and 2.6%, between 2.6% and 2.7%, between 2.7% and 2.8%, between 2.8% and 2.9%, between 2.9% and 3%, between 3% and 3.1%, between 3.1% and 3.2%, between 3.2% and 3.3%, between 3.3% and 3.4%, between 3.4% and 3.5%, and between 3.5% and 3.6% on a dry weight basis. In a further aspect, a tobacco plant comprises an average nicotine or total alkaloid level selected from the group consisting of about between 0.01% and 0.1%, between 0.02% and 0.2%, between 0.03% and 0.3%, between 0.04% and 0.4%, between 0.05% and 0.5%, between 0.75% and 1%, between 0.1% and 1.5%, between 0.15% and 2%, between 0.2% and 3%, and between 0.3% and 3.5% on a dry weight basis.

The present disclosure also provides a tobacco plant having an altered nicotine level without negative impacts over other tobacco traits, e.g., leaf grade index value. In an aspect, a low-nicotine or nicotine-free tobacco variety provides cured tobacco of commercially acceptable grade. Tobacco grades are evaluated based on factors including, but not limited to, the leaf stalk position, leaf size, leaf color, leaf uniformity and integrity, ripeness, texture, elasticity, sheen (related with the intensity and the depth of coloration of the leaf as well as the shine), hygroscopicity (the faculty of the tobacco leaves to absorb and to retain the ambient moisture), and green nuance or cast. Leaf grade can be determined, for example, using an Official Standard Grade published by the Agricultural Marketing Service of the US Department of Agriculture (7 U.S.C. § 511). See, e.g., Official Standard Grades for Burley Tobacco (U.S. Type 31 and Foreign Type 93), effective Nov. 5, 1990 (55 F.R. 40645); Official Standard Grades for Flue-Cured Tobacco (U.S. Types 11, 12, 13, 14 and Foreign Type 92), effective Mar. 27, 1989 (54 F.R. 7925); Official Standard Grades for Pennsylvania Seedleaf Tobacco (U.S. Type 41), effective Jan. 8, 1965 (29 F.R. 16854); Official Standard Grades for Ohio Cigar-Leaf Tobacco (U.S. Types 42, 43, and 44), effective Dec. 8, 1963 (28 F.R. 11719 and 28 F.R. 11926); Official Standard Grades for Wisconsin Cigar-Binder Tobacco (U.S. Types 54 and 55), effective Nov. 20, 1969 (34 F.R. 17061); Official Standard Grades for Wisconsin Cigar-Binder Tobacco (U.S. Types 54 and 55), effective Nov. 20, 1969 (34 F.R. 17061); Official Standard Grades for Georgia and Florida Shade-Grown Cigar-Wrapper Tobacco (U.S. Type 62), Effective April 1971. A USDA grade index value can be determined according to an industry accepted grade index. See, e.g., Bowman et al, Tobacco Science, 32:39-40 (1988); Legacy Tobacco Document Library (Bates Document #523267826-523267833, Jul. 1, 1988, Memorandum on the Proposed Burley Tobacco Grade Index); and Miller et al., 1990, Tobacco Intern., 192:55-57 (all foregoing references are incorporated by inference in their entirety). In an aspect, a USDA grade index is a 0-100 numerical representation of federal grade received and is a weighted average of all stalk positions. A higher grade index indicates higher quality. Alternatively, leaf grade can be determined via hyper-spectral imaging. See e.g., WO 2011/027315 (published on Mar. 10, 2011, and incorporated by inference in its entirety).

In an aspect, a tobacco plant provided herein comprises a similar level of one or more tobacco aroma compounds compared to a control tobacco plant when grown in similar growth conditions. In another aspect, a tobacco plant provided herein comprise a similar level of one or more tobacco aroma compounds selected from the group consisting of 3-methylvaleric acid, valeric acid, isovaleric acid, a labdenoid, a cembrenoid, a sugar ester, and a reducing sugar, compared to a control tobacco plant when grown in similar growth conditions.

As used herein, tobacco aroma compounds are compounds associated with the flavor and aroma of tobacco smoke. These compounds include, but are not limited to, 3-methylvaleric acid, valeric acid, isovaleric acid, cembrenoid and labdenoid diterpenes, and sugar esters. Concentrations of tobacco aroma compounds can be measured by any known metabolite profiling methods in the art including, without limitation, gas chromatography mass spectrometry (GC-MS), Nuclear Magnetic Resonance Spectroscopy, liquid chromatography-linked mass spectrometry. See The Handbook of Plant Metabolomics, edited by Weckwerth and Kahl, (Wiley-Blackwell) (May 28, 2013).

As used herein, “reducing sugar(s)” are any sugar (monosaccharide or polysaccharide) that has a free or potentially free aldehyde or ketone group. Glucose and fructose act as nicotine buffers in cigarette smoke by reducing smoke pH and effectively reducing the amount of “free” unprotonated nicotine. Reducing sugars balances smoke flavor, for example, by modifying the sensory impact of nicotine and other tobacco alkaloids. An inverse relationship between sugar content and alkaloid content has been reported across tobacco varieties, within the same variety, and within the same plant line caused by planting conditions. Reducing sugar levels can be measured using a segmented-flow colorimetric method developed for analysis of tobacco samples as adapted by Skalar Instrument Co (West Chester, PA) and described by Davis, Tobacco Science 20:139-144 (1976). For example, a sample is dialyzed against a sodium carbonate solution. Copper neocuproin is added to the sample and the solution is heated. The copper neocuproin chelate is reduced in the presence of sugars resulting in a colored complex which is measured at 460 nm.

In an aspect, a tobacco plant comprises one or more non-naturally existing mutant alleles in one or more QPT gene loci which reduce or eliminate QPT enzymatic activity from the one or more OPT gene loci. In an aspect, these mutant alleles result in lower nicotine levels. Mutant qpt alleles can be introduced by any method known in the art including random or targeted mutagenesis approaches.

Such mutagenesis methods include, without limitation, treatment of seeds with ethyl methylsulfate (EMS) (Hildering and Verkerk, In, The use of induced mutations in plant breeding. Pergamon press, pp 317-320, 1965) or UV-irradiation, X-rays, and fast neutron irradiation (see, for example, Verkerk, Neth. J. Agric. Sci. 19:197-203, 1971; and Poehlman, Breeding Field Crops, Van Nostrand Reinhold, New York (3.sup.rd ed), 1987), transposon tagging (Fedoroff et al., 1984; U.S. Pat. Nos. 4,732,856 and 5,013,658), as well as T-DNA insertion methodologies (Hoekema et al., 1983; U.S. Pat. No. 5,149,645). EMS-induced mutagenesis consists of chemically inducing random point mutations over the length of the genome. Fast neutron mutagenesis consists of exposing seeds to neutron bombardment which causes large deletions through double stranded DNA breakage. Transposon tagging comprises inserting a transposon within an endogenous gene to reduce or eliminate expression of the gene. The types of mutations that may be present in a tobacco gene include, for example, point mutations, deletions, insertions, duplications, and inversions. Such mutations desirably are present in the coding region of a tobacco gene; however mutations in the promoter region, and intron, or an untranslated region of a tobacco gene may also be desirable.

In addition, a fast and automatable method for screening for chemically induced mutations, TILLING (Targeting Induced Local Lesions In Genomes), using denaturing HPLC or selective endonuclease digestion of selected PCR products is also applicable to the present disclosure. See, McCallum et al. (2000) Nat. Biotechnol. 18:455-457. Mutations that impact gene expression or that interfere with the function of genes can be determined using methods that are well known in the art. Insertional mutations in gene exons usually result in null-mutants. Mutations in conserved residues can be particularly effective in inhibiting the function of a protein. In an aspect, tobacco plants comprise a nonsense (e.g., stop codon) mutation in one or more QPT genes described herein.

It will be appreciated that, when identifying a mutation, the endogenous reference DNA sequence should be from the same variety of tobacco. For example, if a modified tobacco plant comprising a mutation is from the variety TN90, then the endogenous reference sequence must be the endogenous TN90 sequence, not a homologous sequence from a different tobacco variety (e.g., K326). Similarly, if a modified tobacco cell comprising a mutation is a TN90 cell, then the endogenous reference sequence must be the endogenous TN90 sequence, not a homologous sequence from a tobacco cell from a different tobacco variety (e.g., K326).

In an aspect, the present disclosure also provides a tobacco line with altered nicotine levels while maintaining commercially acceptable leaf quality. This line can be produced by introducing mutations into one or more OPT genes via precise genome engineering technologies, for example, Transcription activator-like effector nucleases (TALENs), meganuclease, zinc finger nuclease, and a clustered regularly-interspaced short palindromic repeats (CRISPR)/Cas9 system, a CRISPR/Cpf1 system, a CRISPR/Csm1 system, and a combination thereof (see, for example, U.S. Patent Application publication 2017/0233756). See, e.g., Gaj et al., Trends in Biotechnology, 31 (7): 397-405 (2013).

The screening and selection of mutagenized tobacco plants can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454), enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

In an aspect, a tobacco plant or plant genome provided herein is mutated or edited by a nuclease selected from the group consisting of a meganuclease, a zinc-finger nuclease (ZFN), a transcription activator-like effector nuclease (TALEN), a CRISPR/Cas9 nuclease, a CRISPR/Cpf1 nuclease, or a CRISPR/Csm1 nuclease.

As used herein, “editing” or “genome editing” refers to targeted mutagenesis of at least 1, at least 2, at least 3, at least 4, at least 5, at least 6, at least 7, at least 8, at least 9, or at least 10 nucleotides of an endogenous plant genome nucleic acid sequence, or removal or replacement of an endogenous plant genome nucleic acid sequence. In an aspect, an edited nucleic acid sequence provided has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with an endogenous nucleic acid sequence. In an aspect, an edited nucleic acid sequence provided has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with a polynucleotide selected from the group consisting of SEQ ID NOs: 1 to 8, and fragments thereof. In another aspect, an edited nucleic acid sequence provided has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with a polynucleotide encoding a polypeptide selected from the group consisting of SEQ ID NOs: 9 to 12.

Meganucleases, ZFNs, TALENs, CRISPR/Cas9, CRISPR/Csm1 and CRISPR/Cpf1 induce a double-strand DNA break at a target site of a genomic sequence that is then repaired by the natural processes of homologous recombination (HR) or non-homologous end-joining (NHEJ). Sequence modifications then occur at the cleaved sites, which can include deletions or insertions that result in gene disruption in the case of NHEJ, or integration of donor nucleic acid sequences by HR. In an aspect, a method provided comprises editing a plant genome with a nuclease provided to mutate at least 1, at least 2, at least 3, at least 4, at least 5, at least 6, at least 7, at least 8, at least 9, at least 10, or more than 10 nucleotides in the plant genome via HR with a donor polynucleotide. In an aspect, a mutation provided is caused by genome editing using a nuclease. In another aspect, a mutation provided is caused by non-homologous end-joining or homologous recombination.

Meganucleases, which are commonly identified in microbes, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

ZFNs are synthetic proteins consisting of an engineered zinc finger DNA-binding domain fused to the cleavage domain of the FokI restriction endonuclease. ZFNs can be designed to cleave almost any long stretch of double-stranded DNA for modification of the zinc finger DNA-binding domain. ZFNs form dimers from monomers composed of a non-specific DNA cleavage domain of FokI endonuclease fused to a zinc finger array engineered to bind a target DNA sequence.

The DNA-binding domain of a ZFN is typically composed of 3-4 zinc-finger arrays. The amino acids at positions −1, +2, +3, and +6 relative to the start of the zinc finger ∞-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art.

The FokI nuclease domain requires dimerization to cleave DNA and therefore two ZFNs with their C-terminal regions are needed to bind opposite DNA strands of the cleavage site (separated by 5-7 bp). The ZFN monomer can cute the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

Without being limited by any scientific theory, because the DNA-binding specificities of zinc finger domains can in principle be re-engineered using one of various methods, customized ZFNs can theoretically be constructed to target nearly any gene sequence. Publicly available methods for engineering zinc finger domains include Context-dependent Assembly (CoDA), Oligomerized Pool Engineering (OPEN), and Modular Assembly.

TALENs are artificial restriction enzymes generated by fusing the transcription activator-like effector (TALE) DNA binding domain to a FokI nuclease domain. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site.

Transcription activator-like effectors (TALEs) can be engineered to bind practically any DNA sequence. TALE proteins are DNA-binding domains derived from various plant bacterial pathogens of the genus Xanthomonas. The Xanthomonas pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

A relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al,. Nucleic Acids Research (2012) 40: W117-122.; Cermak et al., Nucleic Acids Research (2011). 39: e82; and tale-nt.cac.cornell.edu/about.

A CRISPR/Cas9 system, CRISPR/Csm1, or a CRISPR/Cpf1 system are alternatives to the FokI-based methods ZFN and TALEN. The CRISPR systems are based on RNA-guided engineered nucleases that use complementary base pairing to recognize DNA sequences at target sites.

CRISPR/Cas9, CRISPR/Csm1, and a CRISPR/Cpf1 systems are part of the adaptive immune system of bacteria and archaea, protecting them against invading nucleic acids such as viruses by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trans-activating CRISPR RNA (tracrRNA) to activate and guide the Cas9 nuclease. This cleaves homologous double-stranded DNA sequences known as protospacers in the invading DNA. A prerequisite for cleavage is the presence of a conserved protospacer-adjacent motif (PAM) downstream of the target DNA, which usually has the sequence 5-NGG-3 but less frequently NAG. Specificity is provided by the so-called “seed sequence” approximately 12 bases upstream of the PAM, which must match between the RNA and target DNA. Cpf1 and Csm1 act in a similar manner to Cas9, but Cpf1 and Csm1 do not require a tracrRNA.

In still another aspect, a tobacco plant provided here comprises one or more qpt mutations and further comprises one or more mutations in one or more loci encoding a nicotine demethylase (e.g., CYP82E4, CYP82E5, CYP82E10) that confer reduced amounts of nornicotine (See U.S. Pat. Nos. 8,319,011; 8,124,851; 9,187,759; 9,228,194; 9,228,195; 9,247,706) compared to a control plant lacking one or more mutations in one or more loci encoding a nicotine demethylase. In an aspect, a tobacco plant described further comprises reduced nicotine demethylase activity compared to a control plant when grown and cured under comparable conditions.

In an aspect, a qpt mutant tobacco plant further comprises a mutation capable of producing a leaf comprising an anabasine level less than the anabasine level of a leaf from a wild-type control tobacco plant when grown and processed under comparable conditions. In another aspect, a qpt mutant tobacco plant further comprises a mutation capable of producing a leaf comprising an anabasine level less than 5%, 10%, 20%, 25%, 30%, 40%, 50%, 60%, 70%, or 80% of the anabasine level of a leaf from a wild-type control tobacco plant when grown and processed under comparable conditions.

In an aspect, a qpt mutant tobacco plant comprises a further mutation capable of producing a leaf comprising a more than two-fold reduction of the anatabine level compared to a leaf from a control tobacco plant when grown and processed under comparable conditions. In another aspect, a qpt mutant tobacco plant comprises a further mutation capable of producing a leaf comprising a more than 3-, 4-, 5-, 6-, 7-, 8-, 9-, 10-, 11-, 12-, or 13-fold reduction of the anatabine level compared to a leaf from a wild-type control tobacco plant when grown and processed under comparable conditions. In an aspect, a mutation providing lower level of anatabine is a mutation described in US Application Publication No. 2014/0283165 and US Application Publication No. 2016/0010103. In another aspect, a qpt mutant further comprises a mutation in a PMT gene. In a further aspect, a qpt mutant plant further comprises a transgene or mutation suppressing the expression or activity of a PMT gene.

In an aspect, a qpt mutant tobacco plant further comprises a mutation capable of providing a nornicotine level less than 80%, 75%, 70%, 65%, 60%, 55%, 50%, 45%, 40%, or 35% of the nornicotine level of a leaf from a wild-type control tobacco plant when grown and processed under comparable conditions.

In an aspect, a qpt mutant tobacco plant is capable of producing a cured leaf comprising a total N-nitrosonornicotine (NNN) level of less than 2, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.6, less than 0.5, less than 0.4, less than 0.3, less than 0.2, less than 0.15, less than 0.1, or less than 0.05 ppm.

In another aspect, a qpt mutant tobacco plant is capable of producing a cured leaf comprising a total NNN level of between 2 and 0.05, between 1.9 and 0.05, between 1.8 and 0.05, between 1.7 and 0.05, between 1.6 and 0.05, between 1.5 and 0.05, between 1.4 and 0.05, between 1.3 and 0.05, between 1.2 and 0.05, between 1.1 and 0.05, between 1.0 and 0.05, between 0.9 and 0.05, between 0.8 and 0.05, between 0.7 and 0.05, between 0.6 and 0.05, between 0.5 and 0.05, between 0.4 and 0.05, between 0.3 and 0.05, between 0.2 and 0.05, between 0.15 and 0.05, or between 0.1 and 0.05 ppm.

In an aspect, a qpt mutant tobacco plant is capable of producing a cured leaf comprising a total nicotine-derived nitrosamine ketone (NNK) level of less than 2, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.6, less than 0.5, less than 0.4, less than 0.3, less than 0.2, less than 0.15, less than 0.1, or less than 0.05 ppm.

In another aspect, a qpt mutant tobacco plant is capable of producing a cured leaf comprising a total NNK level of between 2 and 0.05, between 1.9 and 0.05, between 1.8 and 0.05, between 1.7 and 0.05, between 1.6 and 0.05, between 1.5 and 0.05, between 1.4 and 0.05, between 1.3 and 0.05, between 1.2 and 0.05, between 1.1 and 0.05, between 1.0 and 0.05, between 0.9 and 0.05, between 0.8 and 0.05, between 0.7 and 0.05, between 0.6 and 0.05, between 0.5 and 0.05, between 0.4 and 0.05, between 0.3 and 0.05, between 0.2 and 0.05, between 0.15 and 0.05, or between 0.1 and 0.05 ppm.

In an aspect, a qpt mutant tobacco plant further comprises a mutation or transgene providing an increased level of one or more antioxidants. In another aspect, a qpt mutant tobacco plant further comprises a genetic modification in an endogenous gene and further comprises an increased level of one or more antioxidants in a cured leaf compared to a control cured tobacco leaf lacking the genetic modification, where the endogenous gene encodes an antioxidant biosynthetic enzyme, a regulatory transcription factor of an antioxidant, an antioxidant transporter, an antioxidant metabolic enzyme, or a combination thereof. In a further aspect, a qpt mutant tobacco plant further comprises a transgene and further comprises an increased level of one or more antioxidants in a cured leaf compared to a control cured tobacco leaf lacking the transgene, where the transgene encodes or directly modulates an antioxidant biosynthetic enzyme, a regulatory transcription factor of an antioxidant, an antioxidant transporter, an antioxidant metabolic enzyme, or a combination thereof. In an aspect, a qpt mutant tobacco plant further comprises a transgene or a cisgenic construct expressing one or more genes selected from the group consisting of AtPAP1, NtAN2, NtAN1, NtJAF13, NtMyb3, chorismate mutase, and arogenate dehydratase (ADT). In another aspect, a qpt mutant tobacco plant further comprises one or more transgenes or genetic modification for increasing antioxidants or decreasing one or more TSNAs as described in WIPO Publication No. 2018/067985 or US Publication No. 2018/0119163.

In an aspect, a tobacco plant described is a modified tobacco plant. As used herein, “modified”, in the context of a plant, refers to a plant comprising a genetic alteration introduced for certain purposes and beyond natural polymorphisms.

In an aspect, a tobacco plant described is a cisgenic plant. As used herein, “cisgenesis” or “cisgenic” refers to genetic modification of a plant, plant cell, or plant genome in which all components (e.g., promoter, donor nucleic acid, selection gene) have only plant origins (i.e., no non-plant origin components are used). In an aspect, a plant, plant cell, or plant genome provided is cisgenic. Cisgenic plants, plant cells, and plant genomes provided can lead to ready-to-use tobacco lines. In another aspect, a tobacco plant provided comprises no non-tobacco genetic material or sequences.

As used herein, “gene expression” or expression of a gene refers to the biosynthesis or production of a gene product, including the transcription and/or translation of the gene product.

In an aspect, a tobacco plant provided comprises one or more qpt mutations and further comprises reduced expression or activity of one or more genes involved in nicotine biosynthesis or transport. Genes involved in nicotine biosynthesis include, but are not limited to, arginine decarboxylase (ADC), methylputrescine oxidase (MPO), NADH dehydrogenase, ornithine decarboxylase (ODC), phosphoribosylanthranilate isomerase (PRAI), quinolate phosphoribosyl transferase (QPT), and S-adenosyl-methionine synthetase (SAMS). Nicotine Synthase, which catalyzes the condensation step between a nicotinic acid derivative and methylpyrrolinium cation, has not been elucidated although two candidate genes (A622 and NBB1) have been proposed. See US 2007/0240728 A1 and US 2008/0120737A1. A622 encodes an isoflavone reductase-like protein. In addition, several transporters may be involved in the translocation of nicotine. A transporter gene, named MATE, has been cloned and characterized (Morita et al., PNAS 106:2447-52 (2009)).

In an aspect, a tobacco plant provided comprises one or more qpt mutations and further comprises a reduced level of mRNA, protein, or both of one or more genes encoding a product selected from the group consisting of MPO, QPT, ADC, ODC, PRAI, SAMS, BBL, MATE, A622, and NBB1, compared to a control tobacco plant. In another aspect, a tobacco plants provided comprises one or more qpt mutations and further comprises a transgene directly suppressing the expression of one or more genes encoding a product selected from the group consisting of MPO, QPT, ADC, ODC, PRAI, SAMS, BBL, MATE, A622, and NBB1. In another aspect, a tobacco plant provided comprises one or more qpt mutations and further comprises a transgene or mutation suppressing the expression or activity of one or more genes encoding a product selected from the group consisting of MPO, QPT, ADC, ODC, PRAI, SAMS, BBL, MATE, A622, and NBB1.

In an aspect, a tobacco plant provided is from a tobacco type selected from the group consisting of flue-cured tobacco, air-cured tobacco, dark air-cured tobacco, dark fire-cured tobacco, Galpao tobacco, and Oriental tobacco. In another aspect, a tobacco plant provided is from a tobacco type selected from the group consisting of Burley tobacco, Maryland tobacco, and dark tobacco.

Flue-cured tobaccos (also called “Virginia” or “bright” tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the United States of America. In one aspect, tobacco plants or seeds or modified tobacco plants or seeds provided herein are of a flue-cured tobacco variety selected from the group consisting of the varieties listed in Table 1, and any variety essentially derived from any one of the foregoing varieties. See WO 2004/041006 A1. In a further aspect, modified tobacco plants or seeds provided herein are in a flue-cured variety selected from the group consisting of K326, K346, and NC196.

TABLE 1
Flue-cured Tobacco Varieties

| | | |
|---|---|---|
| 400 (TC 225) | K 346 | Reams 134 |
| 401 (TC 226) | K 346 (TC 569) | Reams 158 |
| 401 Cherry Red (TC 227) | K 358 | Reams 713 |
| 401 Cherry Red | K 394 (TC 321) | Reams 744 |
| Free (TC 228) | K 399 | Reams M1 |
| Cash (TC 250) | K 399 (TC 322) | RG 11 (TC 600) |
| Cash (TI 278) | K 730 | RG 13 (TC 601) |
| CC 101 | Lonibow (TI 1573) | RG 17 (TC 627) |
| CC 1063 | Lonibow (TI 1613) | RG 22 (TC 584) |
| CC 13 | McNair 10 (TC 330) | RG 8 (TC 585) |
| CC 143 | McNair 135 | RG 81 (TC 618) |
| CC 200 | (TC 337) | RG H51 |
| CC 27 | McNair 30 (TC 334) | RG4H 217 |
| CC 301 | McNair 373 | RGH 12 |
| CC 33 | (TC 338) | RGH 4 |
| CC 35 | McNair 944 | RGH 51 |
| CC 37 | (TC 339) | RGH 61 |
| CC 400 | MK94 (TI 1512) | SC 58 (TC 400) |
| CC 500 | MS K 326 | SC 72 (TC 403) |
| CC 600 | MS NC 71 | Sp. G-168 |
| CC 65 | MS NC 72 | SPEIGHT 168 |
| CC 67 | NC 100 | Speight 168 (TC 633) |
| CC 700 | NC 102 | Speight 172 (TC 634) |
| CC 800 | NC 1071 (TC 364) | Speight 178 |
| CC 900 | NC 1125-2 | Speight 179 |
| Coker 139 (TC 259) | NC 12 (TC 346) | Speight 190 |
| Coker 139 yb1, yb2 | NC 1226 | Speight 196 |
| Coker 140 (TC 260) | NC 196 | SPEIGHT 220 |
| Coker 176 (TC 262) | NC 2326 (TC 365) | SPEIGHT 225 |
| Coker 187 (TC 263) | NC 27 NF (TC 349) | SPEIGHT 227 |
| Coker 187-Hicks | NC 291 | SPEIGHT 236 |
| (TC 265) | NC 297 | Speight G-10 |
| Coker 209 (TC 267) | NC 299 | (TC 416) |
| Coker 258 (TC 270) | NC 37 NF (TC 350) | Speight G-102 |
| Coker 298 (TC 272) | NC 471 | Speight G-108 |
| Coker 316 (TC 273) | NC 55 | Speight G-111 |
| Coker 319 (TC 274) | NC 567 (TC 362) | Speight G-117 |
| Coker 347 (TC 275) | NC 60 (TC 352) | Speight G-126 |
| Coker 371-Gold | NC 606 | Speight G-15 |
| (TC 276) | NC 6140 | (TC 418) |
| Coker 411 (TC 277) | NC 71 | Speight G-23 |
| Coker 48 (TC 253) | NC 72 | Speight G-28 |
| Coker 51 (TC 254) | NC 729 (TC 557) | (TC 420) |
| Coker 86 (TC 256) | NC 810 (TC 659) | Speight G-33 |
| CU 263 (TC 619) | NC 82 (TC 356) | Speight G-41 |
| CU 561 | NC 8640 | Speight G-5 |
| DH95-1562-1 | NC 89 (TC 359) | Speight G-52 |
| Dixie Bright 101 | NC 92 | Speight G-58 |
| (TC 290) | NC 925 | Speight G-70 |
| Dixie Bright 102 | NC 95 (TC 360) | Speight G-70 |
| (TC 291) | NC 98 (TC 361) | (TC 426) |
| Dixie Bright 244 | NC EX 24 | Speight G-80 |
| (TC 292) | NC PY 10 (TC 367) | (TC 427) |
| Dixie Bright 27 | NC TG 61 | Speight NF3 |
| (TC 288) | Oxford 1 (TC 369) | (TC 629) |
| Dixie Bright 28 | Oxford 1-181 | STNCB |
| (TC 289) | (TC 370) | VA 182 |
| GF 157 | Oxford 2 (TC 371) | VA 45 (TC 559) |
| GF 318 | Oxford 207 | Vesta 30 (TC 439) |
| GL 26H | (TC 632) | Vesta 33 (TC 440) |
| GL 338 | Oxford 26 | Vesta 5 (TC 438) |
| GL 350 | (TC 373) | Vesta 62 (TC 441) |
| GL 368 | Oxford 3 (TC 372) | Virginia (TI 220) |
| GL 395 | Oxford 414 NF | Virginia (TI 273) |
| GL 600 | PD 611 (TC 387) | Virginia (TI 877) |
| GL 737 | PVH 03 | Virginia 115 |
| GL 939 | | (TC 444) |
| GL 939 (TC 628) | PVH 09 | Virginia 21 (TC 443) |
| Hicks (TC 310) | PVH 1118 | Virginia Bright |
| Hicks Broadleaf | PVH 1452 | (TI 964) |
| (TC 311) | | Virginia Bright |
| K 149 (TC 568) | PVH 1600 | Leaf (TC 446) |
| K 317 | PVH 2110 | Virginia Gold |
| K 326 | PVH 2275 | (TC 447) |
| K 326 (TC 319) | R 83 (Line 256-1) | White Stem |
| K 340 (TC 320) | (TI 1400) | Orinoco (TC 451) |

Air-cured tobaccos include “Burley,” “Maryland,” and “dark” tobaccos. The common factor linking air-cured tobaccos is that curing occurs primarily without artificial sources of heat and humidity. Burley tobaccos are light to dark brown in color, high in oil, and low in sugar. Burley tobaccos are typically air-cured in barns. Major Burley growing countries include Argentina, Brazil, Italy, Malawi, and the United States of America.

Maryland tobaccos are extremely fluffy, have good burning properties, low nicotine and a neutral aroma. Major Maryland growing countries include the United States of America and Italy.

In one aspect, tobacco plants or seeds or modified tobacco plants or seeds provided herein are of a Burley tobacco variety selected from the group consisting of the tobacco varieties listed in Table 2, and any variety essentially derived from any one of the foregoing varieties. In a further aspect, modified tobacco plants or seeds provided herein are in a Burley variety selected from the group consisting of TN 90, KT 209, KT 206, KT212, and HB 4488.

TABLE 2
Burley Tobacco Varieties

| | | |
|---|---|---|
| 4407 LC | HB 4108P | KY 54 (TC 71) |
| AA-37-1 | HB 4151P | KY 56 (TC 72) |
| Burley 21 (TC 7) | HB 4192P | KY 56 (TC 72) |
| Burley 49 (TC 10) | HB 4194P | KY 57 (TC 73) |
| Burley 64 (TC 11) | HB 4196 | KY 58 (TC 74) |
| Burley Mammoth | HB 4488 | KY 8654 (TC 77) |
| KY 16 (TC 12) | HB 4488P | KY 8959 |
| Clay 402 | HB04P | KY 9 (TC 54) |
| Clay 403 | HB 4488 LC | KY 907 LC |
| Clay 502 | HIB 21 | KY 908 (TC 630) |
| Clays 403 | HPB 21 | NBH 98 |
| GR 10 (TC 19) | HY 403 | (Screened) |
| GR 10 (TC 19) | Hybrid 403 LC | NC 1206 |
| GR 10A (TC 20) | Hybrid 404 LC | NC 129 |
| GR 13 (TC 21) | Hybrid 501 LC | NC 2000 LC |
| GR 14 (TC 22) | KDH-959 | NC 2002 LC |
| GR 149 LC | (TC 576) | NC 3 LC |
| GR 153 | KDH-960 | NC 5 LC |
| GR 17 (TC 23) | (TC 577) | NC 6 LC |
| GR 17B (TC 24) | KT 200 LC | NC 7 LC |
| GR 18 (TC 25) | KT 204 LC | NC BH 129 LC |
| GR 19 (TC 26) | KT 206 LC | NC03-42-2 |
| GR 2 (TC 15) | KT 209 LC | Newton 98 |
| GR 24 (TC 27) | KT 210 LC | R 610 LC |
| GR 36 (TC 28) | KT 212 LC | R 630 LC |
| GR 38 (TC 29) | KT 215 LC | R 7-11 |
| GR 38A (TC 30) | KY 1 (TC 52) | R 7-12 LC |
| GR 40 (TC 31) | KY 10 (TC 55) | RG 17 |
| GR 42 (TC 32) | KY 12 (TC 56) | TKF 1801 LC |
| GR 42C (TC 33) | KY 14 (TC 57) | TKF 2002 LC |
| GR 43 (TC 34) | KY 14 × L8 LC | TKF 4024 LC |
| GR 44 (TC 35) | KY 15 (TC 58) | TKF 4028 LC |
| GR 45 (TC 36) | KY 16 (TC 59) | TKF 6400 LC |
| GR 46 (TC 37) | KY 17 (TC 60) | TKF 7002 LC |
| GR 48 (TC 38) | KY 19 (TC 61) | TKS 2002 LC |
| GR 5 (TC 16) | KY 21 (TC 62) | TN 86 (TC 82) |
| GR 53 (TC 39) | KY 22 (TC 63) | TN 90 LC |
| GR 6 (TC 17) | KY 24 (TC 64) | TN 97 Hybrid LC |
| GR 9 (TC 18) | KY 26 (TC 65) | TN 97 LC |
| GR139 NS | KY 33 (TC 66) | VA 116 |
| GR139 S | KY 34 (TC 67) | VA 119 |
| HB 04P | KY 35 (TC 68) | Virgin A Mutante |
| HB 04P LC | KY 41A (TC 69) | (TI 1406) |
| HB 3307P LC | KY 5 (TC 53) | Virginia 509 |
| | KY 52 (TC 70) | (TC 84) |

In another aspect, tobacco plants or seeds or modified tobacco plants or seeds provided herein are of a Maryland tobacco variety selected from the group consisting of the tobacco varieties listed in Table 3, and any variety essentially derived from any one of the foregoing varieties.

TABLE 3
Maryland Tobacco Varieties

Maryland 10 (TC 498)
Maryland 14 D2 (TC 499)
Maryland 201 (TC 503)
Maryland 21 (TC 500)
Maryland 341 (TC 504)
Maryland 40
Maryland 402
Maryland 59 (TC 501)
Maryland 601
Maryland 609 (TC 505)
Maryland 64 (TC 502)
Maryland 872 (TC 506)
Maryland Mammoth (TC 507)

Dark air-cured tobaccos are distinguished from other tobacco types primarily by its curing process, which gives dark air-cured tobacco its medium-brown to dark-brown color and a distinct aroma. Dark air-cured tobaccos are mainly used in the production of chewing tobacco and snuff. In one aspect, modified tobacco plants or seeds provided herein are of a dark air-cured tobacco variety selected from the group consisting of Sumatra, Jatim, Dominican Cubano, Besuki, One sucker, Green River, Virginia sun-cured, and Paraguan Passado, and any variety essentially derived from any one of the foregoing varieties.

Dark fire-cured tobaccos are generally cured with low-burning wood fires on the floors of closed curing barns. Dark fire-cured tobaccos are typically used for making pipe blends, cigarettes, chewing tobacco, snuff, and strong-tasting cigars. Major growing regions for dark fire-cured tobaccos are Tennessee, Kentucky, and Virginia in the United States of America. In one aspect, tobacco plants or seeds or modified tobacco plants or seeds provided herein are of a dark fire-cured tobacco variety selected from the group consisting of the tobacco varieties listed in Table 4, and any variety essentially derived from any one of the foregoing varieties.

TABLE 4
Dark Fire-Cured Tobacco Varieties

| | | |
|---|---|---|
| Black Mammoth | KY 171 (TC 475) | PD 7309 LC |
| (TC 461) | KY 171 LC | PD 7312 LC |
| Black Mammoth Small | KY 171 NS | |
| Stalk (TC 641) | KY 180 (TC 573) | PD 7318 LC |
| Certified Madole | KY 190 (TC 574) | PD 7319 LC |
| (TC 463) | Little Crittenden | Petico M PG04 |
| D-534-A-1 (TC 464) | Little Crittenden | PY KY 160 |
| DAC ULT 302 | (TC 476) | (TC 612) |
| DAC ULT 303 | Little Crittenden | PY KY 171 |
| DAC ULT 306 | LC (certified) | (TC 613) |
| DAC ULT 308 | Little Crittenden PhPh | Shirey |
| DAC ULT 312 | Lizard Tail | TI 1372 |
| DF 300 (TC 465) | Turtle Foot | TN D94 |
| DF 485 (TC 466) | Madole (TC 478) | TN D94 (TC 621) |
| DF 516 (TC 467) | Madole (TC 479) | TN D950 |
| DF 911 (TC 468) | MS KY 171 | TN D950 (PhPh) |
| DT 508 | MS NL Madole LC | TN D950 |
| DT 518 (Screened) | MS TN D950 LC | TN D950 (TC 622) |
| DT 538 LC | Nance (TC 616) | TR Madole |
| DT 592 | Narrow Leaf Madole | (TC 486) |
| Improved Madole | LC (certified) | VA 309 |
| (TC 471) | Neal Smith Madole | VA 309 (TC 560) |
| Jernigan's Madole | (TC 646) | VA 309 LC |
| (TC 472) | Newtons VH Madole | (certified) |
| KT 14LC | NL Madole | VA 310 (TC 487) |
| KT D17LC | NL Madole (PhPh) | VA 331 (TC 592) |
| KT D4 LC | NL Madole (TC 484) | VA 355 (TC 638) |
| KT D6 LC | NL Madole LC | VA 359 |
| KT D8 LC | NL Madole LC (PhPh) | VA 359 (Screened) |
| KY 153 (TC 216) | NL Madole NS | VA 359 (TC 639) |
| KY 157 (TC 217) | One Sucker (TC 224) | VA 359 LC |
| KY 160 | OS 400 | (certified) |
| KY 160 (TC 218) | PD 302H | VA 403 (TC 580) |
| KY 163 (TC 219) | PD 312H | VA 405 (TC 581) |
| KY 165 (TC 220) | PD 318H | VA 409 (TC 562) |
| KY 170 (TC 474) | PD 7302 LC | VA 510 (TC 572) |
| KY 171 (PhPh) | PD 7305 | |

Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant size, small leaf size, and unique aroma properties of Oriental tobacco varieties are a result of their adaptation to the poor soil and stressful climatic conditions in which they have been developed. In one aspect, tobacco plants or seeds or modified tobacco plants or seeds provided herein are of an Oriental tobacco variety selected from the group consisting of the tobacco varieties listed in Table 5, and any variety essentially derived from any one of the foregoing varieties.

TABLE 5
Oriental Tobacco Varieties

| | | |
|---|---|---|
| Bafra (TI 1641) | Edirne (TI 1671) | Samsun (TC 536) |
| Bahce (TI 1730) | Ege (TI 1642) | Samsun 959 (TI 1570) |
| Bahia (TI 1416) | Ege-64 (TI 1672) | Samsun Evkaf (TI 1723) |
| Bahia (TI 1455) | Izmir (Akhisar) | Samsun Holmes NN |
| Baiano (TI 128) | (TI 1729) | (TC 540) |
| Basma | Izmir (Gavurkoy) | Samsun Maden (TI 1647) |
| Basma (TI 1666) | (TI 1727) | Samsun NO 15 (TC 541) |
| Basma Drama | Izmir Ege 64 | Samsun-BLK SHK |
| Basma Hybrid | Izmir-Incekara | Tol (TC 542) |
| (PhPh) | (TI 1674) | Samsun-Canik (TI 1678) |
| Basma Zihna I | Izmir-Ozbas (TI 1675) | Samsun-Maden (TI 1679) |
| Bitlis (TI 1667) | Jaka Dzebel (TI 1326) | Saribaptar |
| Bitlis (TI 1725) | Kaba-Kulak | 407-Izmir Region |
| Bubalovac (TI 1282) | Kagoshima Maruba | Smyrna (TC 543) |
| Bursa (TI 1650) | (TI 158) | Smyrna No. 23 (TC 545) |
| Bursa (TI 1668) | Katerini | Smyrna No. 9 (TC 544) |
| Canik (TI 1644) | Katerini S53 | Smyrna-Blk Shk Tol |
| Djebel 174 (TI 1492) | Krumovgrad 58 | (TC 546) |
| Djebel 359 (TI 1493) | MS Basma | Trabzon (TI 1649) |
| Djebel 81 | MS Katerini S53 | Trabzon (TI 1682) |
| Dubec 566 (TI 1409) | Nevrokop 1146 | Trapezund 161 (TI 1407) |
| Dubec 7 (TI 1410) | Ozbas (TI 1645) | Turkish (TC 548) |
| Dubek 566 (TI 1567) | Perustitza (TI 980) | Turkish Angshit (TI 90) |
| Duzce (TI 1670) | Prilep (TI 1291) | Turkish Samsum (TI 92) |
| | Prilep (TI 1325) | Turkish Tropizoid (TI 93) |
| | Prilep 12-2/1 | Turkish Varotic (TI 89) |
| | Prilep 23 | Xanthi (TI 1662) |

In an aspect, tobacco plants or seeds or modified tobacco plants or seeds provided herein are of an cigar tobacco variety selected from the group consisting of the tobacco varieties listed in Table 6, and any variety essentially derived from any one of the foregoing varieties.

TABLE 6
Cigar Tobacco Varieties

| | | |
|---|---|---|
| Bahai (TI 62) | Castillo Negro, Blanco, | Enshu (TI 1586) |
| Beinhart 1000 | Pina (TI 449) | Florida 301 |
| Beinhart 1000 (TI 1562) | Caujaro (TI 893) | Florida 301 (TC 195) |
| Beinhart 1000-1 | Chocoa (TI 289) | PA Broadleaf |
| (TI 1561) | Chocoa (TI 313) | (TC 119) |
| Bergerac C | Connecticut 15 | Pennsylvania |
| Bergerac C | (TC 183) | Broadleaf |
| (TI 1529) | Connecticut | Pennsylvania |
| Big Cuban (TI 1565) | Broadleaf | Broadleaf (TC 119) |
| Castillo Negro, Blanco, | Connecticut Broadleaf | Petite Havana SR1 |
| Pina (TI 448) | (TC 186) | Petite Havana |
| Castillo Negro, Blanco, | Connecticut Shade | SR1 (TC 105) |
| Pina (TI 448A) | (TC 188) | |
| | Criollo, Colorado | |
| | (TI 1093) | |

In an aspect, tobacco plants or seeds or modified tobacco plants or seeds provided herein are of a tobacco variety selected from the group consisting of the tobacco varieties listed in Table 7, and any variety essentially derived from any one of the foregoing varieties.

TABLE 7
Other Tobacco Varieties

Chocoa (TI 319)
Hoja Parada (TI 1089)
Hoja Parado (Galpoa) (TI 1068)
Perique (St. James Parrish)
Perique (TC 556)
Perique (TI 1374)
Sylvestris (TI 984)
TI 179

In an aspect, a tobacco plant, seed, or cell is from a variety selected from the group consisting of the tobacco varieties listed in Table 1, Table 2, Table 3, Table 4, Table 5, Table 6, and Table 7.

In an aspect, low-alkaloid or low-nicotine tobacco plants, seeds, hybrids, varieties, or lines are essentially derived from or in the genetic background of a variety selected from the group consisting of the tobacco varieties listed in Table 1, Table 2, Table 3, Table 4, Table 5, Table 6, and Table 7.

All foregoing mentioned specific varieties of flue-cured, dark air-cured, Burley, Maryland, dark fire-cured, cigar, or Oriental type are listed only for exemplary purposes. Any additional flue-cured, dark air-cured, Burley, Maryland, dark fire-cured, cigar, or Oriental varieties are also contemplated in the present application.

Also provided are populations of tobacco plants described. In an aspect, a population of tobacco plants has a planting density of between about 5,000 and about 8000, between about 5,000 and about 7,600, between about 5,000 and about 7,200, between about 5,000 and about 6,800, between about 5,000 and about 6,400, between about 5,000 and about 6,000, between about 5,000 and about 5,600, between about 5,000 and about 5,200, between about 5,200 and about 8,000, between about 5,600 and about 8,000, between about 6,000 and about 8,000, between about 6,400 and about 8,000, between about 6,800 and about 8,000, between about 7,200 and about 8,000, or between about 7,600 and about 8,000 plants per acre. In another aspect, a population of tobacco plants is in a soil type with low to medium fertility.

Also provided are containers of seeds from tobacco plants described. A container of tobacco seeds of the present disclosure may contain any number, weight, or volume of seeds. For example, a container can contain at least, or greater than, about 100, 200, 300, 400, 500, 600, 700, 800, 900, 1000, 1500, 2000, 2500, 3000, 3500, 4000 or more seeds. Alternatively, the container can contain at least, or greater than, about 1 ounce, 5 ounces, 10 ounces, 1 pound, 2 pounds, 3 pounds, 4 pounds, 5 pounds or more seeds. Containers of tobacco seeds may be any container available in the art. By way of non-limiting example, a container may be a box, a bag, a packet, a pouch, a tape roll, a tube, or a bottle.

Also provided is cured tobacco material made from a low-alkaloid or low-nicotine tobacco plant described. Further provided is cured tobacco material made from a tobacco plant described with higher levels of total alkaloid or nicotine.

“Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. In an aspect, green leaf tobacco provided can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation. Fermentation typically is characterized by high initial moisture content, heat generation, and a 10 to 20% loss of dry weight. See, e.g., U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cure, aged, and fermented tobacco can be further processed (e.g., cut, shredded, expanded, or blended). See, for example, U.S. Pat. Nos. 4,528,993; 4,660,577; and 4,987,907. In an aspect, the cured tobacco material of the present disclosure is sun-cured. In another aspect, the cured tobacco material of the present disclosure is flue-cured, air-cured, or fire-cured.

Tobacco material obtained from the tobacco lines, varieties or hybrids of the present disclosure can be used to make tobacco products. As used herein, “tobacco product” is defined as any product made or derived from tobacco that is intended for human use or consumption.

Tobacco products provided include, without limitation, cigarette products (e.g., cigarettes and bidi cigarettes), cigar products (e.g., cigar wrapping tobacco and cigarillos), pipe tobacco products, products derived from tobacco, tobacco-derived nicotine products, smokeless tobacco products (e.g., moist snuff, dry snuff, and chewing tobacco), films, chewables, tabs, shaped parts, gels, consumable units, insoluble matrices, hollow shapes, reconstituted tobacco, expanded tobacco, and the like. See, e.g., U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokable material includes cured tobacco within a tobacco blend. In an aspect, a tobacco product of the present disclosure is selected from the group consisting of a cigarillo, a non-ventilated recess filter cigarette, a vented recess filter cigarette, a cigar, snuff, pipe tobacco, cigar tobacco, cigarette tobacco, chewing tobacco, leaf tobacco, hookah tobacco, shredded tobacco, and cut tobacco. In another aspect, a tobacco product of the present disclosure is a smokeless tobacco product. Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat treated smokeless tobacco. Dry snuff is finely ground tobacco that is placed in the mouth or used nasally. In a further aspect, a tobacco product of the present disclosure is selected from the group consisting of loose leaf chewing tobacco, plug chewing tobacco, moist snuff, and nasal snuff. In yet another aspect, a tobacco product of the present disclosure is selected from the group consisting of an electronically heated cigarette, an e-cigarette, an electronic vaporing device.

In an aspect, a tobacco product of the present disclosure can be a blended tobacco product. In another aspect, a tobacco product of the present disclosure can be a low nicotine tobacco product. In a further aspect, a tobacco product of the present disclosure may comprise nornicotine at a level of less than about 3 mg/g. For example, the nornicotine content in such a product can be 3.0 mg/g, 2.5 mg/g, 2.0 mg/g, 1.5 mg/g, 1.0 mg/g, 750 pg/g, 500 pg/g, 250 pg/g, 100 pg/g, 75 pg/g, 50 pg/g, 25 pg/g, 10 pg/g, 7.0 pg/g, 5.0 pg/g, 4.0 pg/g, 2.0 pg/g, 1.0 pg/g, 0.5 pg/g, 0.4 pg/g, 0.2 pg/g, 0.1 pg/g, 0.05 pg/g, 0.01 pg/g, or undetectable.

In an aspect, cured tobacco material or tobacco products provided comprise an average nicotine or total alkaloid level selected from the group consisting of about 0.01%, 0.02%, 0.05%, 0.75%, 0.1%, 0.15%, 0.2%, 0.3%, 0.35%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, and 9% on a dry weight basis. In another aspect, cured tobacco material or tobacco products provided comprise an average nicotine or total alkaloid level selected from the group consisting of about between 0.01% and 0.02%, between 0.02% and 0.05%, between 0.05% and 0.75%, between 0.75% and 0.1%, between 0.1% and 0.15%, between 0.15% and 0.2%, between 0.2% and 0.3%, between 0.3% and 0.35%, between 0.35% and 0.4%, between 0.4% and 0.5%, between 0.5% and 0.6%, between 0.6% and 0.7%, between 0.7% and 0.8%, between 0.8% and 0.9%, between 0.9% and 1%, between 1% and 1.1%, between 1.1% and 1.2%, between 1.2% and 1.3%, between 1.3% and 1.4%, between 1.4% and 1.5%, between 1.5% and 1.6%, between 1.6% and 1.7%, between 1.7% and 1.8%, between 1.8% and 1.9%, between 1.9% and 2%, between 2% and 2.1%, between 2.1% and 2.2%, between 2.2% and 2.3%, between 2.3% and 2.4%, between 2.4% and 2.5%, between 2.5% and 2.6%, between 2.6% and 2.7%, between 2.7% and 2.8%, between 2.8% and 2.9%, between 2.9% and 3%, between 3% and 3.1%, between 3.1% and 3.2%, between 3.2% and 3.3%, between 3.3% and 3.4%, between 3.4% and 3.5%, and between 3.5% and 3.6% on a dry weight basis. In a further aspect, cured tobacco material or tobacco products provided comprise an average nicotine or total alkaloid level selected from the group consisting of about between 0.01% and 0.1%, between 0.02% and 0.2%, between 0.03% and 0.3%, between 0.04% and 0.4%, between 0.05% and 0.5%, between 0.75% and 1%, between 0.1% and 1.5%, between 0.15% and 2%, between 0.2% and 3%, and between 0.3% and 3.5% on a dry weight basis.

The present disclosure also provides methods for breeding tobacco lines, cultivars, or varieties comprising a desirable level of total alkaloid or nicotine, e.g., low nicotine or nicotine free. Breeding can be carried out via any known procedures. DNA fingerprinting, SNP mapping, haplotype mapping or similar technologies may be used in a marker-assisted selection (MAS) breeding program to transfer or breed a desirable trait or allele into a tobacco plant. For example, a breeder can create segregating populations in a F₂ or backcross generation using F1 hybrid plants or further crossing the F1 hybrid plants with other donor plants with an agronomically desirable genotype. Plants in the F₂ or backcross generations can be screened for a desired agronomic trait or a desirable chemical profile using one of the techniques known in the art or listed herein. Depending on the expected inheritance pattern or the MAS technology used, self-pollination of selected plants before each cycle of backcrossing to aid identification of the desired individual plants can be performed. Backcrossing or other breeding procedure can be repeated until the desired phenotype of the recurrent parent is recovered. A recurrent parent in the present disclosure can be a flue-cured variety, a Burley variety, a dark air-cured variety, a dark fire-cured variety, or an Oriental variety. Other breeding techniques can be found, for example, in Wernsman, E. A., and Rufty, R. C. 1987. Chapter Seventeen. Tobacco. Pages 669-698 In: Cultivar Development. Crop Species. W.H. Fehr (ed.), MacMillan Publishing Go., Inc., New York, N.Y., incorporated herein by reference in their entirety.

Results of a plant breeding program using the tobacco plants described includes useful lines, cultivars, varieties, progeny, inbreds, and hybrids of the present disclosure. As used herein, the term “variety” refers to a population of plants that share constant characteristics which separate them from other plants of the same species. A variety is often, although not always, sold commercially. While possessing one or more distinctive traits, a variety is further characterized by a very small overall variation between individuals within that variety. A “pure line” variety may be created by several generations of self-pollination and selection, or vegetative propagation from a single parent using tissue or cell culture techniques. A variety can be essentially derived from another line or variety. As defined by the International Convention for the Protection of New Varieties of Plants (Dec. 2, 1961, as revised at Geneva on Nov. 10, 1972; on Oct. 23, 1978; and on Mar. 19, 1991), a variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it conforms to the initial variety in the expression of the essential characteristics that result from the genotype or combination of genotypes of the initial variety. Essentially derived varieties can be obtained, for example, by the selection of a natural or induced mutant, a somaclonal variant, a variant individual from plants of the initial variety, backcrossing, or transformation. A first tobacco variety and a second tobacco variety from which the first variety is essentially derived, are considered as having essentially identical genetic background. A “line” as distinguished from a variety most often denotes a group of plants used non-commercially, for example in plant research. A line typically displays little overall variation between individuals for one or more traits of interest, although there may be some variation between individuals for other traits.

In an aspect, this disclosure provides a tobacco plant, variety, line, or cell comprising one or more qpt mutations provided in any one of Tables 10A, 10B, 11A, and 11B.

In another aspect, this disclosure provides a tobacco plant, variety, line, or cell derived from any tobacco plant, variety, or line provided in any one of Tables 11A and 11B.

In an aspect, this disclosure provides the tobacco line 19GH655 and F1 or F2 tobacco plants, or male sterile tobacco plants, derived therefrom. In an aspect, this disclosure provides the tobacco line 19GH756 and F₁ or F₂ tobacco plants, or male sterile tobacco plants, derived therefrom. In an aspect, this disclosure provides the tobacco line 19GH752 and F₁ or F₂ tobacco plants, or male sterile tobacco plants, derived therefrom. In an aspect, this disclosure provides the tobacco line 19GH753 and F₁ or F₂ tobacco plants, or male sterile tobacco plants, derived therefrom. In an aspect, this disclosure provides the tobacco line 19GH754 and F₁ or F₂ tobacco plants, or male sterile tobacco plants, derived therefrom. In an aspect, this disclosure provides the tobacco line 19GH755 and F₁ or F₂ tobacco plants, or male sterile tobacco plants, derived therefrom. In an aspect, this disclosure provides the tobacco line 19GH749 and F₁ or F₂ tobacco plants, or male sterile tobacco plants, derived therefrom. In an aspect, this disclosure provides the tobacco line 19GH971 and F₁ or F₂ tobacco plants, or male sterile tobacco plants, derived therefrom. In an aspect, this disclosure provides the tobacco line 19GH757 and F₁ or F₂ tobacco plants, or male sterile tobacco plants, derived therefrom. In an aspect, this disclosure provides the tobacco line 19GH972 and F₁ or F₂ tobacco plants, or male sterile tobacco plants, derived therefrom. In an aspect, this disclosure provides the tobacco line 19GH973 and F₁ or F₂ tobacco plants, or male sterile tobacco plants, derived therefrom. In an aspect, this disclosure provides the tobacco line 19GH750 and F₁ or F₂ tobacco plants, or male sterile tobacco plants, derived therefrom. In an aspect, this disclosure provides the tobacco line 19GH796 and F₁ or F₂ tobacco plants, or male sterile tobacco plants, derived therefrom. In an aspect, this disclosure provides the tobacco line 19GH975 and F₁ or F₂ tobacco plants, or male sterile tobacco plants, derived therefrom. In an aspect, this disclosure provides the tobacco line 19GH661 and F₁ or F₂ tobacco plants, or male sterile tobacco plants, derived therefrom. In an aspect, this disclosure provides the tobacco line 19GH662 and F₁ or F₂ tobacco plants, or male sterile tobacco plants, derived therefrom. In an aspect, this disclosure provides the tobacco line 19GH663 and F₁ or F₂ tobacco plants, or male sterile tobacco plants, derived therefrom. In an aspect, this disclosure provides the tobacco line 19GH974 and F₁ or F₂ tobacco plants, or male sterile tobacco plants, derived therefrom. In an aspect, this disclosure provides the tobacco line 19GH352 and F₁ or F₂ tobacco plants, or male sterile tobacco plants, derived therefrom. In an aspect, this disclosure provides the tobacco line 19GH353 and F₁ or F₂ tobacco plants, or male sterile tobacco plants, derived therefrom. In an aspect, this disclosure provides the tobacco line 19GH354 and F₁ or F₂ tobacco plants, or male sterile tobacco plants, derived therefrom. In an aspect, this disclosure provides the tobacco line 19GH797 and F₁ or F₂ tobacco plants, or male sterile tobacco plants, derived therefrom. In an aspect, this disclosure provides the tobacco line 19GH759 and F₁ or F₂ tobacco plants, or male sterile tobacco plants, derived therefrom. In an aspect, this disclosure provides the tobacco line 19GH798 and F₁ or F₂ tobacco plants, or male sterile tobacco plants, derived therefrom. In an aspect, this disclosure provides the tobacco line 19GH1025 and F₁ or F₂ tobacco plants, or male sterile tobacco plants, derived therefrom. In an aspect, this disclosure provides the tobacco line 19GH355 and F₁ or F₂ tobacco plants, or male sterile tobacco plants, derived therefrom. In an aspect, this disclosure provides the tobacco line 19GH351 and F₁ or F₂ tobacco plants, or male sterile tobacco plants, derived therefrom. In an aspect, this disclosure provides the tobacco line 19GH744 and F₁ or F₂ tobacco plants, or male sterile tobacco plants, derived therefrom. In an aspect, this disclosure provides the tobacco line 19GH746 and F₁ or F₂ tobacco plants, or male sterile tobacco plants, derived therefrom. In an aspect, this disclosure provides the tobacco line 19GH744 and F₁ or F₂ tobacco plants, or male sterile tobacco plants, derived therefrom. In an aspect, this disclosure provides the tobacco line 19GH760 and F₁ or F₂ tobacco plants, or male sterile tobacco plants, derived therefrom.

In an aspect, the present disclosure provides a method of introgressing a low nicotine trait into a tobacco variety, the method comprising: (a) crossing a first tobacco variety comprising a low nicotine trait with a second tobacco variety without the low nicotine trait to produce one or more progeny tobacco plants; (b) genotyping the one or more progeny tobacco plants for a qpt mutant allele selected from those listed in Tables 11A and 11B; and (c) selecting a progeny tobacco plant comprising the qpt mutant allele. In another aspect, these methods further comprise backcrossing the selected progeny tobacco plant with the second tobacco variety. In a further aspect, these methods further comprise: (d) crossing the selected progeny plant with itself or with the second tobacco variety to produce one or more further progeny tobacco plants; and (e) selecting a further progeny tobacco plant comprising a low nicotine trait. In an aspect, the step (e) of selecting comprises marker-assisted selection. In an aspect, these methods produce a single gene conversion comprising a low nicotine trait. In an aspect, these methods produce a single gene conversion comprising a qpt mutant allele. In an aspect, the second tobacco variety is an elite variety. In another aspect, the genotyping step of these methods involve one or more molecular marker assays. In another aspect, the genotyping may involve a polymorphic marker comprising a polymorphism selected from the group consisting of single nucleotide polymorphisms (SNPs), insertions or deletions in DNA sequence (Indels), simple sequence repeats of DNA sequence (SSRs), a restriction fragment length polymorphism (RFLP), and a tag SNP.

As used herein, “locus” is a chromosomal locus or region where a polymorphic nucleic acid, trait determinant, gene, or marker is located. A “locus” can be shared by two homologous chromosomes to refer to their corresponding locus or region. As used herein, “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant allele for a gene may have a reduced or eliminated activity or expression level for the gene relative to the wild-type allele. For diploid organisms such as tobacco, a first allele can occur on one chromosome, and a second allele can occur at the same locus on a second homologous chromosome. If one allele at a locus on one chromosome of a plant is a mutant allele and the other corresponding allele on the homologous chromosome of the plant is wild-type, then the plant is described as being heterozygous for the mutant allele. However, if both alleles at a locus are mutant alleles, then the plant is described as being homozygous for the mutant alleles. A plant homozygous for mutant alleles at a locus may comprise the same mutant allele or different mutant alleles if heteroallelic or biallelic.

As used herein, “introgression” or “introgress” refers to the transmission of a desired allele of a genetic locus from one genetic background to another.

As used herein, “crossed” or “cross” means to produce progeny via fertilization (e.g. cells, seeds or plants) and includes crosses between plants (sexual) and self-fertilization (selfing).

As used herein, “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. The initial cross gives rise to the F1 generation. The term “BC1” refers to the second use of the recurrent parent, “BC2” refers to the third use of the recurrent parent, and so on. In an aspect, a backcross is performed repeatedly, with a progeny individual of each successive backcross generation being itself backcrossed to the same parental genotype.

As used herein, “single gene converted” or “single gene conversion” refers to plants that are developed using a plant breeding technique known as backcrossing, or via genetic engineering, where essentially all of the desired morphological and physiological characteristics of a variety are recovered in addition to the single gene transferred into the variety via the backcrossing technique or via genetic engineering.

As used herein, “elite variety” means any variety that has resulted from breeding and selection for superior agronomic performance.

As used herein, “selecting” or “selection” in the context of marker-assisted selection or breeding refer to the act of picking or choosing desired individuals, normally from a population, based on certain pre-determined criteria.

As used herein, the term “trait” refers to one or more detectable characteristics of a cell or organism which can be influenced by genotype. The phenotype can be observable to the naked eye, or by any other means of evaluation known in the art, e.g., microscopy, biochemical analysis, genomic analysis, an assay for a particular disease tolerance, etc. In some cases, a phenotype is directly controlled by a single gene or genetic locus, e.g., a “single gene trait.” In other cases, a phenotype is the result of several genes.

As used herein, “marker assay” means a method for detecting a polymorphism at a particular locus using a particular method, e.g., measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc.

As used herein, “marker assisted selection” (MAS) is a process by which phenotypes are selected based on marker genotypes. “Marker assisted selection breeding” refers to the process of selecting a desired trait or traits in a plant or plants by detecting one or more nucleic acids from the plant, where the nucleic acid is linked to the desired trait, and then selecting the plant or germplasm possessing those one or more nucleic acids.

As used herein, “polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism (RFLP), and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

As used herein, “SNP” or “single nucleotide polymorphism” means a sequence variation that occurs when a single nucleotide (A, T, C, or G) in the genome sequence is altered or variable. “SNP markers” exist when SNPs are mapped to sites on the genome.

As used herein, “marker” or “molecular marker” or “marker locus” is a term used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest. Each marker is therefore an indicator of a specific segment of DNA, having a unique nucleotide sequence. The map positions provide a measure of the relative positions of particular markers with respect to one another. When a trait is stated to be linked to a given marker it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage and/or under environmental conditions where the trait can be expressed.

It is understood that any tobacco plant of the present disclosure can further comprise additional agronomically desirable traits, for example, by transformation with a genetic construct or transgene using a technique known in the art. Without limitation, an example of a desired trait is herbicide resistance, pest resistance, disease resistance; high yield; high grade index value; curability; curing quality; mechanical harvestability; holding ability; leaf quality; height, plant maturation (e.g., early maturing, early to medium maturing, medium maturing, medium to late maturing, or late maturing); stalk size (e.g., a small, medium, or a large stalk); or leaf number per plant (e.g., a small (e.g., 5-10 leaves), medium (e.g., 11-15 leaves), or large (e.g., 16-21) number of leaves), or any combination. In an aspect, low-nicotine or nicotine-free tobacco plants or seeds disclosed comprise one or more transgenes expressing one or more insecticidal proteins, such as, for example, a crystal protein of Bacillus thuringiensis or a vegetative insecticidal protein from Bacillus cereus, such as VIP3 (see, for example, Estruch et al. (1997) Nat. Biotechnol. 15:137). In another aspect, tobacco plants further comprise an introgressed trait conferring resistance to brown stem rot (U.S. Pat. No. 5,689,035) or resistance to cyst nematodes (U.S. Pat. No. 5,491,081).

The present disclosure also provides qpt mutant tobacco plants comprising an altered nicotine or total alkaloid level but having a yield comparable to the yield of corresponding initial tobacco plants without such a nicotine level alternation. In an aspect, a qpt mutant tobacco variety provides a yield selected from the group consisting of about between 1200 and 3500, between 1300 and 3400, between 1400 and 3300, between 1500 and 3200, between 1600 and 3100, between 1700 and 3000, between 1800 and 2900, between 1900 and 2800, between 2000 and 2700, between 2100 and 2600, between 2200 and 2500, and between 2300 and 2400 lbs/acre. In another aspect, a qpt mutant tobacco variety provides a yield selected from the group consisting of about between 1200 and 3500, between 1300 and 3500, between 1400 and 3500, between 1500 and 3500, between 1600 and 3500, between 1700 and 3500, between 1800 and 3500, between 1900 and 3500, between 2000 and 3500, between 2100 and 3500, between 2200 and 3500, between 2300 and 3500, between 2400 and 3500, between 2500 and 3500, between 2600 and 3500, between 2700 and 3500, between 2800 and 3500, between 2900 and 3500, between 3000 and 3500, and between 3100 and 3500 lbs/acre. In a further aspect, qpt mutant tobacco plants provide a yield between 65% and 130%, between 70% and 130%, between 75% and 130%, between 80% and 130%, between 85% and 130%, between 90% and 130%, between 95% and 130%, between 100% and 130%, between 105% and 130%, between 110% and 130%, between 115% and 130%, or between 120% and 130% of the yield of a control plant having essentially identical genetic background except for qpt mutation(s). In a further aspect, qpt mutant tobacco plants provide a yield between 70% and 125%, between 75% and 120%, between 80% and 115%, between 85% and 110%, or between 90% and 100% of the yield of a control plant having essentially identical genetic background except for qpt mutation(s).

In an aspect, a tobacco plant disclosed (e.g., a low-nicotine, nicotine-free, or low-alkaloid tobacco variety) comprises a modification conferring a desired trait (e.g., low-nicotine, nicotine-free, or low-alkaloid) without substantially impacting a trait selected from the group consisting of yield, ripening and senescence, susceptibility to insect herbivory, polyamine content after topping, chlorophyll level, mesophyll cell number per unit leaf area, and end-product quality after curing.

In an aspect, a tobacco plant disclosed comprises a modification conferring a desired trait (e.g., low-nicotine, nicotine-free, or low-alkaloid) and further comprises a trait substantially comparable to an unmodified control plant, where the trait is selected from the group consisting of yield, ripening and senescence, susceptibility to insect herbivory, polyamine content after topping, chlorophyll level, mesophyll cell number per unit leaf area, and end-product quality after curing.

In an aspect, a tobacco plant disclosed comprises a modification conferring a desired trait (e.g., low-nicotine, nicotine-free, or low-alkaloid) and further comprises a yield which is more than 80%, more than 85%, more than 90%, more than 95%, more than 100%, more than 105%, more than 110%, more than 115%, more than 120%, more than 125%, more than 130%, more than 135%, or more than 140% relative to the yield of an unmodified control plant. In an aspect, a tobacco plant disclosed comprises a modification conferring a desired trait (e.g., low-nicotine, nicotine-free, or low-alkaloid) and further comprises a yield which is between 70% and 140%, between 75% and 135%, between 80% and 130%, between 85% and 125%, between 90% and 120%, between 95% and 115%, or between 100% and 110% relative to the yield of an unmodified control plant. In an aspect, a tobacco plant disclosed comprises a modification conferring a desired trait (e.g., low-nicotine, nicotine-free, or low-alkaloid) and further comprises a yield which is between 70% and 80%, between 75% and 85%, between 80% and 90%, between 85% and 95%, between 90% and 100%, between 95% and 105%, between 105% and 115%, between 110% and 120%, between 115% to 125%, between 120% and 130%, between 125 and 135%, or between 130% and 140% relative to the yield of an unmodified control plant.

In an aspect, a low-nicotine or nicotine-free tobacco variety disclosed is adapted for machine harvesting. In another aspect, a low-nicotine or nicotine-free tobacco variety disclosed is harvested mechanically.

In an aspect, a tobacco plant comprising at least one mutant allele in a qpt gene comprises a low-nicotine trait. In another aspect, a tobacco plant comprising at least one mutant allele in a qpt gene comprises a nicotine-free trait. In another aspect, a tobacco plant comprising at least one mutant allele in a qpt gene comprises a low-alkaloid trait.

In an aspect, tobacco plants provided are hybrid plants. Hybrids can be produced by preventing self-pollination of female parent plants (e.g., seed parents) of a first variety, permitting pollen from male parent plants of a second variety to fertilize the female parent plants, and allowing F1 hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by male sterility (MS), or transgenic male sterility where a transgene inhibits microsporogenesis and/or pollen formation, or self-incompatibility. Female parent plants containing MS are particularly useful. In aspects in which the female parent plants are MS, pollen may be harvested from male fertile plants and applied manually to the stigmas of MS female parent plants, and the resulting F1 seed is harvested.

Plants can be used to form single-cross tobacco F1 hybrids. Pollen from a male parent plant is manually transferred to an emasculated female parent plant or a female parent plant that is male sterile to form F1 seed. Alternatively, three-way crosses can be carried out where a single-cross F1 hybrid is used as a female parent and is crossed with a different male parent. As another alternative, double-cross hybrids can be created where the F1 progeny of two different single-crosses are themselves crossed. Self-incompatibility can be used to particular advantage to prevent self-pollination of female parents when forming a double-cross hybrid.

In an aspect, a low-nicotine or nicotine-free tobacco variety is male sterile. In another aspect, a low-nicotine or nicotine-free tobacco variety is cytoplasmic male sterile. Male sterile tobacco plants may be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W.H. Fehr (ed.), MacMillan Publishing Go., Inc., New York, N.Y. 761 pp.

In an aspect, this disclosure provides a male sterile tobacco plant, variety, or line comprising one or more qpt mutations provided in any one of Tables 11A and 11B.

In another aspect, this disclosure provides a male sterile tobacco plant, variety, or line derived from any tobacco plant, variety, or line provided in any one of 10A, 10B, 11A, and 11B.

In a further aspect, tobacco parts provided include, but are not limited to, a leaf, a stem, a root, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In an aspect, tobacco part provided does not include seed. In an aspect, this disclosure provides tobacco plant cells, tissues, and organs that are not reproductive material and do not mediate the natural reproduction of the plant. In another aspect, this disclosure also provides tobacco plant cells, tissues, and organs that are reproductive material and mediate the natural reproduction of the plant. In another aspect, this disclosure provides tobacco plant cells, tissues, and organs that cannot maintain themselves via photosynthesis. In another aspect, this disclosure provides somatic tobacco plant cells. Somatic cells, contrary to germline cells, do not mediate plant reproduction.

Cells, tissues and organs can be from seed, fruit, leaf, cotyledon, hypocotyl, meristem, embryos, endosperm, root, shoot, stem, pod, flower, infloresence, stalk, pedicel, style, stigma, receptacle, petal, sepal, pollen, anther, filament, ovary, ovule, pericarp, phloem, vascular tissue. In another aspect, this disclosure provides a tobacco plant chloroplast. In a further aspect, this disclosure provides epidermal cells, stomata cell, leaf or root hairs, a storage root, or a tuber. In another aspect, this disclosure provides a tobacco protoplast.

Skilled artisans understand that tobacco plants naturally reproduce via seeds, not via asexual reproduction or vegetative propagation. In an aspect, this disclosure provides tobacco endosperm. In another aspect, this disclosure provides tobacco endosperm cells. In a further aspect, this disclosure provides a male or female sterile tobacco plant, which cannot reproduce without human intervention.

In an aspect, the present disclosure provides a nucleic acid molecule comprising at least about 40%, 45%, 50%, 55%, 60%, 65%, 70%, 75%, 80%, 85%, 90%, 91%, 92%, 93%, 94%, 95%, 96%, 97%, 98%, 99%, or 100% identity to a sequence selected from the group consisting of SEQ ID NOs: 1 to 8, and fragments thereof. In an aspect, the present disclosure provides a polypeptide or protein comprising at least about 40%, 45%, 50%, 55%, 60%, 65%, 70%, 75%, 80%, 85%, 90%, 91%, 92%, 93%, 94%, 95%, 96%, 97%, 98%, 99%, or 100% identity to an amino acid sequence selected from the group consisting of SEQ ID NOs: 9 to 12.

As used herein, the term “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution.

The present disclosure further provides a method manufacturing a tobacco product comprising tobacco material from tobacco plants disclosed. In an aspect, methods comprise conditioning aged tobacco material made from tobacco plants to increase its moisture content from between about 12.5% and about 13.5% to about 21%, blending the conditioned tobacco material to produce a desirable blend. In an aspect, the method of manufacturing a tobacco product further comprises casing or flavoring the blend. Generally, during the casing process, casing or sauce materials are added to blends to enhance their quality by balancing the chemical composition and to develop certain desired flavor characteristics. Further details for the casing process can be found in Tobacco Production, Chemistry and Technology, Edited by L. Davis and M. Nielsen, Blackwell Science, 1999.

Tobacco material provided can be also processed using methods including, but not limited to, heat treatment (e.g., cooking, toasting), flavoring, enzyme treatment, expansion and/or curing. Both fermented and non-fermented tobaccos can be processed using these techniques. Examples of suitable processed tobaccos include dark air-cured, dark fire cured, burley, flue cured, and cigar filler or wrapper, as well as the products from the whole leaf stemming operation. In an aspect, tobacco fibers include up to 70% dark tobacco on a fresh weight basis. For example, tobacco can be conditioned by heating, sweating and/or pasteurizing steps as described in U.S. Publication Nos. 2004/0118422 or 2005/0178398.

Tobacco material provided can be subject to fermentation. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. In addition to modifying the aroma of the leaf, fermentation can change either or both the color and texture of a leaf. Also during the fermentation process, evolution gases can be produced, oxygen can be taken up, the pH can change, and the amount of water retained can change. See, for example, U.S. Publication No. 2005/0178398 and Tso (1999, Chapter 1 in Tobacco, Production, Chemistry and Technology, Davis & Nielsen, eds., Blackwell Publishing, Oxford). Cured, or cured and fermented tobacco can be further processed (e.g., cut, expanded, blended, milled or comminuted) prior to incorporation into the oral product. The tobacco, in some cases, is long cut fermented cured moist tobacco having an oven volatiles content of between 48 and 50 weight percent prior to mixing with the copolymer and optionally flavorants and other additives.

In an aspect, tobacco material provided can be processed to a desired size. In an aspect, tobacco fibers can be processed to have an average fiber size of less than 200 micrometers. In an aspect, tobacco fibers are between 75 and 125 micrometers. In another aspect, tobacco fibers are processed to have a size of 75 micrometers or less. In an aspect, tobacco fibers include long cut tobacco, which can be cut or shredded into widths of about 10 cuts/inch up to about 110 cuts/inch and lengths of about 0.1 inches up to about 1 inch. Double cut tobacco fibers can have a range of particle sizes such that about 70% of the double cut tobacco fibers falls between the mesh sizes of −20 mesh and 80 mesh.

Tobacco material provided can be processed to have a total oven volatiles content of about 10% by weight or greater; about 20% by weight or greater; about 40% by weight or greater; about 15% by weight to about 25% by weight; about 20% by weight to about 30% by weight; about 30% by weight to about 50% by weight; about 45% by weight to about 65% by weight; or about 50% by weight to about 60% by weight. Those of skill in the art will appreciate that “moist” tobacco typically refers to tobacco that has an oven volatiles content of between about 40% by weight and about 60% by weight (e.g., about 45% by weight to about 55% by weight, or about 50% by weight). As used herein, “oven volatiles” are determined by calculating the percentage of weight loss for a sample after drying the sample in a pre-warmed forced draft oven at 110° C. for 3.25 hours. The oral product can have a different overall oven volatiles content than the oven volatiles content of the tobacco fibers used to make the oral product. The processing steps described can reduce or increase the oven volatiles content.

Having now generally described the disclosure, the same will be more readily understood through reference to the following examples that are provided by way of illustration, and are not intended to be limiting of the present disclosure, unless specified.

### EXAMPLES

### Example 1: Expression Profiling of Four QPT Genes

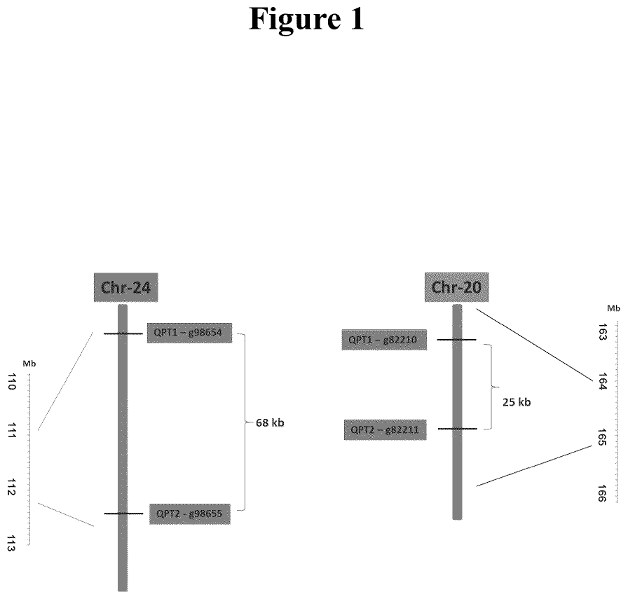

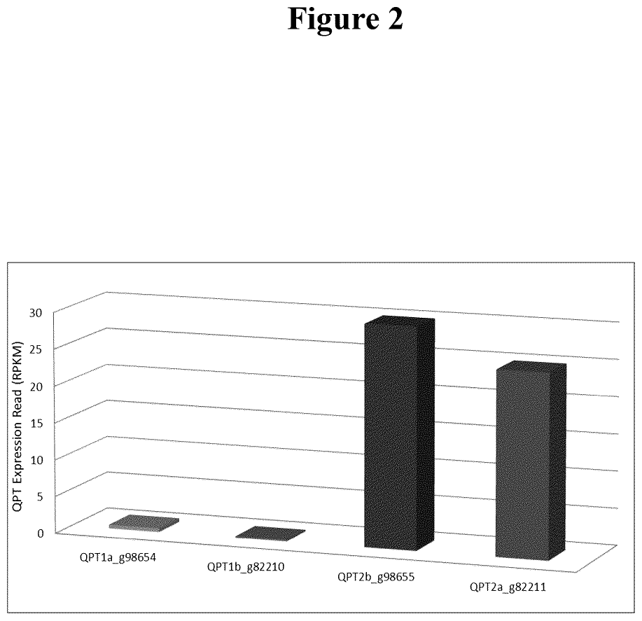

Nicotine biosynthesis involves the formation of nicotinate mononucleotide, which is later converted into the pyridine ring of nicotine. The formation of nicotinate mononucleotide is catalyzed by quinolinate phosphoribosyl transferase (QPT). Depending on the variety, up to four genes encoding QPT (QPT1a, QPT1b, QPT2a, and QPT2b) are present in the tobacco (Nicotiana tabacum) genome (FIG. 1). Table 8A lists genomic DNA sequences, cDNA sequences, and protein sequences of four OPT genes. Tables 8B and 8C provide cDNA and protein sequence identities among four QPT genes. RNA expression levels of four QPT genes in TN90 roots support that, without being limited by any particular theory, QPT2a and QPT2b represent two major QPT genes (FIG. 2).

TABLE 8A
Sequences of four tobacco QPT genes

| | Genomic DNA Sequence | | |
|---|---|---|---|
| | (including regions such | | |
| | as promoter, 5′ UTR, | CDNA | Protein |
| | introns, 3′ UTR, and | Sequence | Sequence |
| | terminator) | (SEQ ID | (SEQ ID |
| Gene Name | (SEQ ID No.) | No.) | No.) |
| QPT1a | 1 | 5 | 9 |
| QPT1b | 2 | 6 | 10 |
| QPT2a | 3 | 7 | 11 |
| QPT2b | 4 | 8 | 12 |

TABLE 8B
cDNA sequence identity among four tobacco
QPT genes determined by Clustal2.1

| cDNA % identity | QPT1a | QPT1b | QPT2a | QPT2b |
|---|---|---|---|---|
| QPT1a | 100 | 97.44 | 92.80 | 93.09 |
| QPT1b | 97.44 | 100 | 93.47 | 93.66 |
| QPT2a | 92.80 | 93.47 | 100 | 98.30 |
| QPT2b | 93.09 | 93.66 | 98.30 | 100 |

TABLE 8C
Protein sequence identity among four tobacco
QPT genes determined by Clustal2.1

| Protein % identity | QPT1a | QPT1b | QPT2a | QPT2b |
|---|---|---|---|---|
| QPT1a | 100 | 97.72 | 90.06 | 91.48 |
| QPT1b | 97.72 | 100 | 91.76 | 92.33 |
| QPT2a | 90.06 | 91.76 | 100 | 98.30 |
| QPT2b | 91.48 | 92.33 | 98.30 | 100 |

TABLE 8D
QPT1a genomic sequence (SEQ ID No. 1) annotation

| Element | Nucleotide Positions |
|---|---|
| 5′ sequence | 1 . . . 544 |
| exon1 | 545 . . . 603 |
| intron1 | 604 . . . 853 |
| exon2 | 854 . . . 987 |
| intron2 | 988 . . . 1649 |
| exon3 | 1650 . . . 1783 |
| intron3 | 1784 . . . 1885 |
| exon4 | 1886 . . . 1949 |
| intron4 | 1950 . . . 2024 |
| exon5 | 2025 . . . 2107 |
| intron5 | 2108 . . . 3209 |
| exon6 | 3210 . . . 3293 |
| intron6 | 3294 . . . 3390 |
| exon7 | 3389 . . . 3541 |
| intron7 | 3542 . . . 4545 |
| exon8 | 4546 . . . 4722 |
| intron8 | 4723 . . . 5114 |
| exon9 | 5115 . . . 5182 |
| intron9 | 5183 . . . 6368 |
| exon10 | 6369 . . . 6465 |
| 3′ sequence | 6466 . . . 6749 |

TABLE 8E
QPT1b genomic sequence (SEQ ID No. 2) annotation

| Element | Nucleotide Positions |
|---|---|
| 5′ sequence | 1 . . . 634 |
| exon1 | 635 . . . 693 |
| intron1 | 694 . . . 953 |
| exon2 | 954 . . . 1087 |
| intron2 | 1086 . . . 1716 |
| exon3 | 1717 . . . 1850 |
| intron3 | 1851 . . . 1951 |
| exon4 | 1952 . . . 2015 |
| intron4 | 2016 . . . 2090 |
| exon5 | 2091 . . . 2173 |
| intron5 | 2174 . . . 3137 |
| exon6 | 3138 . . . 3221 |
| intron6 | 3222 . . . 3316 |
| exon7 | 3317 . . . 3469 |
| intron7 | 3470 . . . 4607 |
| exon8 | 4608 . . . 4784 |
| intron8 | 4785 . . . 4981 |
| exon9 | 4982 . . . 5049 |
| intron9 | 5050 . . . 6044 |
| exon10 | 6045 . . . 6141 |
| 3′ sequence | 6142 . . . 6607 |

TABLE 8F
QPT2a genomic sequence (SEQ ID No. 3) annotation

| Element | Nucleotide Positions |
|---|---|
| 5′ sequence | 1 . . . 493 |
| exon1 | 494 . . . 552 |
| intron1 | 553 . . . 664 |
| exon2 | 665 . . . 801 |
| intron2 | 802 . . . 1602 |
| exon3 | 1603 . . . 1734 |
| intron3 | 1735 . . . 1838 |
| exon4 | 1839 . . . 1902 |
| intron4 | 1903 . . . 1975 |
| exon5 | 1976 . . . 2058 |
| intron5 | 2059 . . . 3156 |
| exon6 | 3157 . . . 3240 |
| intron6 | 3241 . . . 3331 |
| exon7 | 3332 . . . 3484 |
| intron7 | 3485 . . . 4055 |
| exon8 | 4056 . . . 4232 |
| intron8 | 4233 . . . 4524 |
| exon9 | 4525 . . . 4592 |
| intron9 | 4593 . . . 4936 |
| exon10 | 4937 . . . 5033 |
| 3′ sequence | 5034 . . . 5494 |

TABLE 8G
QPT2b genomic sequence (SEQ ID No. 4) annotation

| Element | Nucleotide Positions |
|---|---|
| 5′ sequence | 1 . . . 464 |
| exon1 | 465 . . . 523 |
| intron1 | 524 . . . 634 |
| exon2 | 635 . . . 771 |
| intron2 | 772 . . . 1533 |
| exon3 | 1534 . . . 1667 |
| intron3 | 1668 . . . 1769 |
| exon4 | 1770 . . . 1833 |
| intron4 | 1834 . . . 1906 |
| exon5 | 1907 . . . 1989 |
| intron5 | 1990 . . . 2745 |
| exon6 | 2746 . . . 2829 |
| intron6 | 2830 . . . 2920 |
| exon7 | 2921 . . . 3073 |
| intron7 | 3074 . . . 3532 |
| exon8 | 3533 . . . 3709 |
| intron8 | 3710 . . . 3873 |
| exon9 | 3874 . . . 3941 |
| intron9 | 3942 . . . 4291 |
| exon10 | 4292 . . . 4388 |
| 3′ sequence | 4389 . . . 4762 |

### Example 2: QPT Genome Editing

QPT knockout mutants are produced by editing various OPT genes. Tobacco protoplasts are transfected using polyethylene glycol (PEG) with plasmids encoding a genome editing technology (GET) protein and a specific guide RNA (gRNA) targeting QPT genes at desired positions. Table 9 lists the gRNA sequence used for OPT editing.

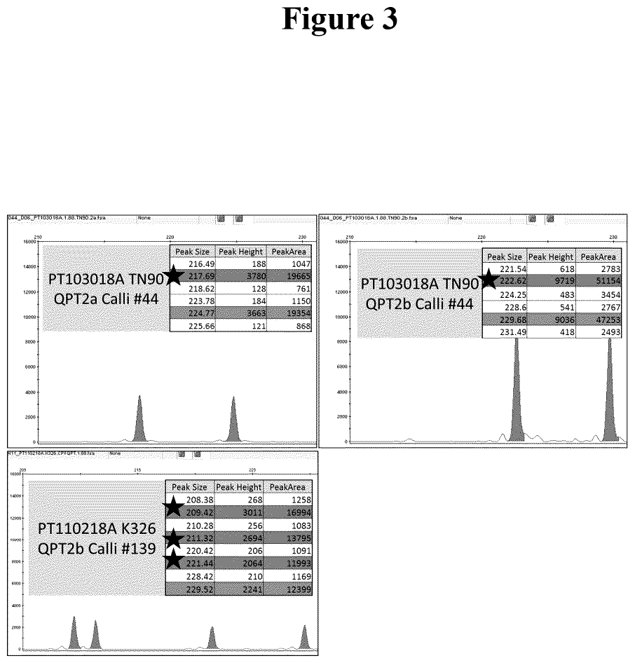

Transfected protoplasts are immobilized in 1% agarose beads and subjected to tissue culture. When calli grow up to ˜1 mm in diameter, they are spread on TOM2 plates. Calli are screened for insertions or deletions (indels) at the target positions using fragment analysis (FIG. 3). Candidates, showing size shifts compared to wildtype control, are selected for further culture. The consequent shoots and rooted shoots are potted and subject to Next Generation Sequencing (NGS) for the target positions to determine the exact deleted sequences.

TABLE 9
gRNA sequences used in the genome editing
technology and its QPT target genes. “Y”
represents that a gRNA targets that QPT gene,
while “—” represents that a gRNA does not
target that QPT gene.

| Genome | | | | | |
|---|---|---|---|---|---|
| Editing | | | | | |
| Tech- | | | | | |
| nology | | | | | |
| (GET) | gRNA sequence | QPT1a | QPT1b | QPT2a | QPT2b |
| gRNA- | TAGCAAAGGAAGACG | — | — | Y | Y |
| QPT1 | GGATCATAG | | | | |
| | (SEQ ID NO: 13) | | | | |

TABLE 10A
Mutant qpt alleles in K326 produced by genome editing using
GET. The position of each edited site (e.g., indels) is
relative to the nucleotide number on the corresponding cDNA
sequence of each QPT gene. For example, line 19GH744 has
tri-allelic mutations in OPT2b. One of the three alleles has
an 8-nucleotide deletion corresponding to nucleotide
positions 269 to 276 of the QPT2b cDNA sequence. The second
allele has a 7-nucleotide deletion corresponding to nucleotide
positions 270 to 276 of the QPT2b cDNA sequence. The
third allele has an 11-nucleotide deletion corresponding
to nucleotide positions 266 to 276 of the QPT2b cDNA
sequence. SEQ ID numbers are assigned and shown for
sequences of 10 or more nucleotides.

| | | QPT2a | | QPT2b | |
|---|---|---|---|---|---|
| | | | Deleted | | Deleted |
| Variety | Line | Position | sequence | Position | sequence |
| K326 | 19GH744 | 268...274 | GGGAT | 269...276 | GGATCATA |
| | | | CA | 270...276 | GATCATA |
| | | | | 266...276 | ACGGGATC |
| | | | | | ATA |
| | | | | | (SEQ ID |
| | | | | | NO: 14) |
| K326 | 19GH746 | 270...274 | GATCA | 269...288 | GGATCATA |
| | | | | | GCAGGGAT |
| | | | | | TGCA |
| | | | | | (SEQ ID |
| | | | | | NO: 15) |
| | | | | 270...277 | GATCATAG |
| K326 | 19GH745 | | | 269...288 | GGATCATA |
| | | | | | GCAGGGATT |
| | | | | | GCA |
| | | | | | (SEQ ID |
| | | | | | NO: 16) |
| K326 | 19GH794 | | | 266...276 | ACGGGATCA |
| | | | | | TA |
| | | | | | (SEQ ID |
| | | | | | NO: 17) |

TABLE 10B
Mutant qpt alleles in TN90 produced by genome editing using GET.
The position of each edited site (e.g., indels) is relative to
the nucleotide number on the corresponding cDNA sequence of each
QPT gene. For example, line 19GH752 has bi-allelic mutations in
QPT2b. One of the two alleles has a 10-nucleotide deletion
corresponding to nucleotide positions 266 to 275 of the QPT2b
cDNA sequence. The other allele has an 8-nucleotide deletion
corresponding to nucleotide positions 268 to 275 of the QPT2b
cDNA sequence. SEQ ID numbers are assigned and shown for sequences
of 10 or more nucleotides.

| | | QPT2a | | QPT2b | |
|---|---|---|---|---|---|
| | | | Deleted | | Deleted |
| Variety | Line | Position | sequence | Position | sequence |
| TN90 | 19GH655 | 268...274 | GGGATCA | | |
| TN90 | 19GH756 | 268...274 | GGGATCA | | |
| TN90 | 19GH752 | 269...271 | GGA | 266...275 | ACGGGAT |
| | | | | | CAT |
| | | | | | (SEQ ID |
| | | | | | NO: 19) |
| | | | | 268...275 | GGGATCA |
| | | | | | T |
| TN90 | 19GH753 | 269...271 | GGA | 266...275 | ACGGGAT |
| | | | | | CAT |
| | | | | | (SEQ ID |
| | | | | | NO: 19) |
| | | | | 268...275 | GGGATCA |
| | | | | | T |
| TN90 | 19GH754 | 269...271 | GGA | 266...275 | ACGGGAT |
| | | | | | CAT |
| | | | | | (SEQ ID |
| | | | | | NO: 19) |
| | | | | 268...275 | GGGATCAT |
| TN90 | 19GH755 | 264...274 | AGACGGG | 269...276 | GGATCATA |
| | | | ATCA | | |
| | | | (SEQ ID | | |
| | | | NO: 18) | | |
| | | 269...274 | GGATCA | 270...274 | GATCA |
| | | 270...275 | GATCAT | | |
| TN90 | 19GH749 | 264...274 | AGACGGG | 269...276 | GGATCATA |
| | | | ATCA | | |
| | | | (SEQ ID | | |
| | | | NO: 18) | | |
| | | 269...274 | GGATCA | 270...274 | GATCA |
| | | 270...275 | GATCAT | | |
| TN90 | 19GH971 | 264... | AGACGGG | 269...276 | GGATCATA |
| | | 274 | ATCA | | |
| | | | (SEQ ID | | |
| | | | NO: 18) | | |
| | | 269...274 | GGATCA | 270...274 | GATCA |
| TN90 | 19GH757 | 269...275 | GGATCAT | 269...275 | GGATCAT |
| TN90 | 19GH972 | 270...275 | GATCAT | 270...274 | GATCA |
| | | | | 274...275 | AT |
| TN90 | 19GH973 | 268...276 | GGGATCA | 268...275 | GGGATCAT |
| | | | TA | 269...276 | GGATCATA |
| TN90 | 19GH750 | 268...275 | GGGATCAT | 269...276 | GGATCATA |
| | | 269...275 | GGATCAT | 270...274 | GATCA |
| TN90 | 19GH796 | 269...276 | GGATCAT | 269...275 | GGATCAT |
| | | | A | | |
| TN90 | 19GH975 | 269...276 | GGATCAT | 269...275 | GGATCAT |
| | | | A | | |
| TN90 | 19GH661 | 268...275 | GGGATCA | 266...274 | ACGGGAT |
| | | | T | | CA |
| | | 270...271 | GA | 270...276 | GATCATA |
| TN90 | 19GH662 | 268...275 | GGGATCA | 266...274 | ACGGGAT |
| | | | T | | CA |
| | | 270...271 | GA | 270...276 | GATCATA |
| TN90 | 19GH663 | 268...275 | GGGATCA | 270...276 | GATCATA |
| | | | T | | |
| | | 270...271 | GA | 266...274 | ACGGGAT |
| | | | | | CA |
| TN90 | 19GH974 | 269...273 | GGATC | | |
| TN90 | 19GH352 | 269...272 | GGAT | 269...276 | GGATCATA |
| TN90 | 19GH353 | 269...272 | GGAT | 269...276 | GGATCATA |
| TN90 | 19GH354 | 269...272 | GGAT | 269...276 | GGATCATA |
| TN90 | 19GH797 | 269...272 | GGAT | 269...276 | GGATCATA |
| TN90 | 19GH759 | 270...274 | GATCA | 269...275 | GGATCAT |
| TN90 | 19GH798 | 270...274 | GATCA | 269...275 | GGATCAT |
| TN90 | 19GH1025 | 270...274 | GATCA | 269...275 | GGATCAT |
| TN90 | 19GH760 | | | 270...276 | GATCATA |
| TN90 | 19GH355 | 270...276 | GATCATA | 269...276 | GGATCATA |
| TN90 | 19GH351 | 270...276 | GATCATA | 269...276 | GGATCATA |
| TN90 | ??? | 270...276 | GATCATA | 269...276 | GGATCATA |

TABLE 11A
Exemplary mutant alleles obtained in the
QPT2a gene

| | | | Deleted |
|---|---|---|---|
| Variety | Line | Position | sequence |
| TN90 | 19GH655 | 268...274 | GGGATCA |
| TN90 | 19GH756 | 268...274 | GGGATCA |
| TN90 | 19GH752 | 269...271 | GGA |
| TN90 | 19GH753 | 269...271 | GGA |
| TN90 | 19GH754 | 269...271 | GGA |
| TN90 | 19GH755 | 264...274 | AGACGGGATCA |
| | | | (SEQ ID NO: 18) |
| | | 269...274 | GGATCA |
| | | 270...275 | GATCAT |
| TN90 | 19GH749 | 264...274 | AGACGGGATCA |
| | | | (SEQ ID NO: 18) |
| | | 269...274 | GGATCA |
| | | 270...275 | GATCAT |
| TN90 | 19GH971 | 264...274 | AGACGGGATCA |
| | | | (SEQ ID NO: 18) |
| | | 269...274 | GGATCA |
| TN90 | 19GH757 | 269...275 | GGATCAT |
| TN90 | 19GH972 | 270...275 | GATCAT |
| TN90 | 19GH973 | 268...276 | GGGATCATA |
| TN90 | 19GH750 | 268...275 | GGGATCAT |
| | | 269...275 | GGATCAT |
| TN90 | 19GH796 | 269...276 | GGATCATA |
| TN90 | 19GH975 | 269...276 | GGATCATA |
| TN90 | 19GH661 | 268...275 | GGGATCAT |
| | | 270...271 | GA |
| TN90 | 19GH662 | 268...275 | GGGATCAT |
| | | 270...271 | GA |
| TN90 | 19GH663 | 268...275 | GGGATCAT |
| | | 270...271 | GA |
| TN90 | 19GH974 | 269...273 | GGATC |
| TN90 | 19GH352 | 269...272 | GGAT |
| TN90 | 19GH353 | 269...272 | GGAT |
| TN90 | 19GH354 | 269...272 | GGAT |
| TN90 | 19GH797 | 269...272 | GGAT |
| TN90 | 19GH759 | 270...274 | GATCA |
| TN90 | 19GH798 | 270...274 | GATCA |
| TN90 | 19GH1025 | 270...274 | GATCA |
| TN90 | 19GH355 | 270...276 | GATCATA |
| TN90 | 19GH351 | 270...276 | GATCATA |
| TN90 | | 270...276 | GATCATA |
| K326 | 19GH744 | 268...274 | GGGATCA |
| K326 | 19GH746 | 270...274 | GATCA |

TABLE 11B
Exemplary mutant alleles obtained in the QPT2b
gene

| | | | Deleted |
|---|---|---|---|
| Variety | Line | Position | sequence |
| TN90 | 19GH752 | 266...275 | ACGGGATCAT |
| | | | (SEQ ID NO: 19) |
| | | 268...275 | GGGATCAT |
| TN90 | 19GH753 | 266...275 | ACGGGATCAT |
| | | | (SEQ ID NO: 19) |
| | | 268...275 | GGGATCAT |
| TN90 | 19GH754 | 266...275 | ACGGGATCAT |
| | | | (SEQ ID NO: 19) |
| | | 268...275 | GGGATCAT |
| TN90 | 19GH755 | 269...276 | GGATCATA |
| | | 270...274 | GATCA |
| TN90 | 19GH749 | 269...276 | GGATCATA |
| | | 270...274 | GATCA |
| TN90 | 19GH971 | 269...276 | GGATCATA |
| | | 270...274 | GATCA |
| TN90 | 19GH757 | 269...275 | GGATCAT |
| TN90 | 19GH972 | 270...274 | GATCA |
| | | 274...275 | AT |
| TN90 | 19GH973 | 268...275 | GGGATCAT |
| | | 269...276 | GGATCATA |
| TN90 | 19GH750 | 269...276 | GGATCATA |
| | | 270...274 | GATCA |
| TN90 | 19GH796 | 269...275 | GGATCAT |
| TN90 | 19GH975 | 269...275 | GGATCAT |
| TN90 | 19GH661 | 266...274 | ACGGGATCA |
| | | 270...276 | GATCATA |
| TN90 | 19GH662 | 266...274 | ACGGGATCA |
| | | 270...276 | GATCATA |
| TN90 | 19GH663 | 270...276 | GATCATA |
| | | 266...274 | ACGGGATCA |
| TN90 | 19GH352 | 269...276 | GGATCATA |
| TN90 | 19GH353 | 269...276 | GGATCATA |
| TN90 | 19GH354 | 269...276 | GGATCATA |
| TN90 | 19GH797 | 269...276 | GGATCATA |
| TN90 | 19GH759 | 269...275 | GGATCAT |
| TN90 | 19GH798 | 269...275 | GGATCAT |
| TN90 | 19GH1025 | 269...275 | GGATCAT |
| TN90 | 19GH760 | 270...276 | GATCATA |
| TN90 | 19GH355 | 269...276 | GGATCATA |
| TN90 | 19GH351 | 269...276 | GGATCATA |
| TN90 | | 269...276 | GGATCATA |
| K326 | 19GH744 | 269...276 | GGATCATA |
| | | 270...276 | GATCATA |
| | | 266...276 | ACGGGATCATA |
| | | | (SEQ ID NO: 14) |
| K326 | 19GH746 | 269...288 | GGATCATAGCAGGGA |
| | | | TTGCA |
| | | | (SEQ ID NO: 15) |
| | | 270...277 | GATCATAG |
| K326 | 19GH745 | 269...288 | GGATCATAGCAGGGA |
| | | | TTGCA |
| | | | (SEQ ID NO: 16) |
| K326 | 19GH794 | 266...276 | ACGGGATCATA |
| | | | (SEQ ID NO: 17) |

### Example 3: Alkaloid Analysis of QPT Edited Lines

Genome edited tobacco plants along with controls are grown in 10″ pots in a greenhouse with 75 PPM fertilizer. At flowering stage, plants are topped and 2 weeks post topping lamina samples were collected from the third, fourth, and fifth leaves from the top of the plant, and alkaloid levels are measured using a method in accordance with CORESTA Method No 62, Determination of Nicotine in Tobacco and Tobacco Products by Gas Chromatographic Analysis, February 2005, and those defined in the Centers for Disease Control and Prevention's Protocol for Analysis of Nicotine, Total Moisture and pH in Smokeless Tobacco Products, as published in the Federal Register Vol. 64, No. 55 Mar. 23, 1999 (and as amended in Vol. 74, No. 4, Jan. 7, 2009).

Briefly, approximately 0.5 g of tobacco is extracted using liquid/liquid extraction into an organic solvent containing an internal standard and analyzed by gas chromatography (GC) with flame ionization detection (FID). Results can be reported as weight percent (Wt %) on either an as is or a dry weight basis. Reporting data on a dry weight basis requires an oven volatiles (OV) determination. Unless specified otherwise, total or individual alkaloid levels or nicotine levels shown herein are on a dry weight basis (e.g., percent total alkaloid or percent nicotine).

Plants are also planted in the field, harvested, and tested for alkaloids and TSNA levels in cured tobacco. Both leaf yield and leaf grade are also assessed for OPT edited plants. Further, different mutant combinations of individual OPT genes are generated and tested (e.g., single, double, triple, or quadruple).

### Example 4: Obtaining Tobacco Lines with Edited Mutant Alleles in One or More QPT Genes

Tobacco lines with mutations in individual QPT genes or selected combinations of QPT genes are obtained from the tobacco lines listed in Tables 10A and 10B. Crossing a single, double, triple, or quadruple mutant (having mutations in one, two, three, or four QPT genes, respectively) to a non-mutated control line and selecting segregating progeny plants for specific QPT mutation combinations. Each mutated gene can be either homozygous or heterozygous for the mutation. Exemplary individual qpt mutant alleles are listed in Tables 11A and 11B.

### Example 5: Further Reduction of Total Alkaloids by Combining Qpt Mutations with Mutations in Other Genes

To further reduce total alkaloids and/or selected individual alkaloids, qpt mutants are combined with mutations in additional genes related to alkaloid biosynthesis in tobacco, such as a quintuple pmt mutant or quinolinate synthase (QS). Briefly, gene editing is used to mutate select pmt or QS genes in a desired mutant background. In the resulting combined pmt/pmt or qs/pmt mutants, alkaloids and TSNA levels are tested in cured tobacco. Both leaf yield and leaf grade are also assessed.

1.-38. (canceled)

39. A tobacco plant, or part thereof, comprising a non-naturally occurring mutant allele of an endogenous QUINOLINATE PHOSPHORIBOSYL TRANSFERASE (QPT) 2a gene, the endogenous QPT2a gene comprising SEQ ID NO: 7, wherein the non-naturally occurring mutant allele comprises a deletion as compared to the endogenous QPT2a gene, and wherein the deletion consists of either a deletion of nucleotide positions 264 to 274 as compared to SEQ ID NO: 7 or a deletion of nucleotide positions 268 to 274 as compared to SEQ ID NO: 7.

40. The tobacco plant, or part thereof, of claim 39, wherein the part thereof is a leaf.

41. Cured tobacco material from the tobacco plant, or part thereof, of claim 39.

42. The cured tobacco material of claim 41, wherein the cured tobacco material is selected from the group consisting of flue-cured tobacco material, air-cured tobacco material, fire-cured tobacco material, and sun-cured tobacco material.

43. A tobacco product comprising the cured tobacco material of claim 41.

44. The tobacco product of claim 43, wherein the tobacco product is selected from the group consisting of a cigarette, a cigarillo, a non-ventilated recess filter cigarette, a vented recess filter cigarette, a cigar, snuff, pipe tobacco, cigar tobacco, cigarette tobacco, chewing tobacco, leaf tobacco, shredded tobacco, and cut tobacco.

45. The tobacco product of claim 43, wherein the tobacco product is a smokeless tobacco product.

46. The tobacco product of claim 45, wherein the smokeless tobacco product is selected from the group consisting of loose leaf chewing tobacco, plug chewing tobacco, moist snuff, and nasal snuff.

47. The tobacco plant, or part thereof, of claim 39, wherein the tobacco plant is from a tobacco type selected from the group consisting of flue-cured tobacco, dark air-cured tobacco, dark fire-cured tobacco, Galpão tobacco, and Oriental tobacco.

48. A tobacco seed from the tobacco plant of claim 39, wherein the tobacco seed comprises the non-naturally occurring mutant allele.

49. A tobacco plant, or part thereof, comprising a non-naturally occurring mutant allele of an endogenous QPT2b gene, the endogenous QPT2b gene comprising SEQ ID NO: 8, wherein the non-naturally occurring mutant allele comprises a deletion as compared to the endogenous QPT2b gene, and wherein the second deletion consists of a deletion of nucleotide positions 269 to 276 as compared to SEQ ID NO: 8.

50. The tobacco plant, or part thereof, of claim 49, wherein the part thereof is a leaf.

51. Cured tobacco material from the tobacco plant, or part thereof, of claim 49.

52. The cured tobacco material of claim 51, wherein the cured tobacco material is selected from the group consisting of flue-cured tobacco material, air-cured tobacco material, fire-cured tobacco material, and sun-cured tobacco material.

53. A tobacco product comprising the cured tobacco material of claim 51.

54. The tobacco product of claim 53, wherein the tobacco product is selected from the group consisting of a cigarette, a cigarillo, a non-ventilated recess filter cigarette, a vented recess filter cigarette, a cigar, snuff, pipe tobacco, cigar tobacco, cigarette tobacco, chewing tobacco, leaf tobacco, shredded tobacco, and cut tobacco.

55. The tobacco product of claim 53, wherein the tobacco product is a smokeless tobacco product.

56. The tobacco product of claim 55, wherein the smokeless tobacco product is selected from the group consisting of loose leaf chewing tobacco, plug chewing tobacco, moist snuff, and nasal snuff.

57. The tobacco plant, or part thereof, of claim 49, wherein the tobacco plant is from a tobacco type selected from the group consisting of flue-cured tobacco, dark air-cured tobacco, dark fire-cured tobacco, Galpão tobacco, and Oriental tobacco.

58. A tobacco seed from the tobacco plant of claim 49, wherein the tobacco seed comprises the non-naturally occurring mutant allele.


SEQUENCE LISTING (22 sequences)

SEQ ID NO: 1 - DNA, 6749 bp, Nicotiana tabacum
  source 1..6749: mol_type = genomic DNA
cctggtaagtcgaactcttagtctttataatttgcaggagcaactctcataagttatatacgattaaacgataacaaacgttataatttagaaaagaaatgccttatttatttttatacatttatttatttatcaccgttgttattgatcttttgtctctgaaccgatattttctgaaaacagtctctctatccctccgggttatggatagagtatgcgtacgttctaccatctcaaaccccaagattattaatggattgttattgttgtacatttatttatttatttatctgcctatcaaatgttgtatcctgtttctttaatttgatatattctgaaaggcatatgttaattgttcgcaaaatcaggaaaagggaagtgaaaaataacctattgtggtcaccctgttaaaccatatagaccaaaacaaccacaaattcacataattggaggactattttgttaaaacaaaagtcttcacagaaagcattaagctccgcaaaagctatttgctacaaaattccttgcataccccaaaatttcaatgtttaaagttcttcctttcactgcaatagtccaccctaatgcaattacagctccaaggtttctttaaaacccctcaatttaacccagaagcaacaacaaaaaaaaggattcttgaaaatcatgtttttatttttgttatatactgataaagattgttcttttatgcaattacagctccaaggtttctttaaaaccactcaattccacccaggaaaaaaaagtcttgaacatcatgtttttattgttttatactgataaagattattcctttatgagtttctgattaaaatttgtttgtaaattgcagattggttgtgaaaatgtcagcaatagccaccaaaaatgcagtggagtcatttgtagtgaagccaccagcacacccaacttatgatttaaagggtgttattcaacttgccctctctgaagatgctggggatataggtttggttttagcttatattgttttttaatcttatgtttgtcaaatgggtttcaataagtttttatacgtggggaaatgctgttaaagaaaactaatggtaaacgttttaattaattgggaggtgctttatgaagtttagctatagtctattttttttttgtgaactccaagcttcattctttcttgtgtagcctcaattagttagagctaattgtgttctttttcgataaggacagctaatgtgttcttaataagtattatcttcagtttaaggaaagaggaggggggaggaatagttctgccaaaagttgctttctttcttgttttccatccgctgtgaggcccaactaatccagattcgcgctaggaagtcccacgttgaggtaaaggtgacttcattctcaggcttgaacgttggacctcttgttaaggattgacgtatcgccccaccacaacctttggcgttaaaagtttttttttttaagttcaatatcaggaggatactgtcatgtactgtctgactctctgcgtgatggaaaagtttgtttgagtaaatttcttggaactagcaatattttcagcacctagctaccattgtgaatttgagtactcaattatacaaaaaccaatttttcctatgtattatttgtgtgaaatgcaggagatgtgacttgtaaggcgacaattcctattgacatggaatccgaagctcattttctagcaaaggaagacgggattgtagcaggaattgcacttgctgagatgatattcgcagaggttgatccttcactaaaggtattgattttttcaattaggttgttctggtaaccggtatttctactttgaagaaatgtctgaaaaaagggttttttaatttcttcatgttctcattatcagatggagtggtctataaatgatggcgataaagttcataaaggcttgaaattcggcaaagtacaaggtaaactatatgtgagaaatgatgaattgctaatgattctttgacttaataataatcttgccttaacgtttgtaggaaaggctcacagcattgttatagctgagagagttgttctcaattttatgcaaaggatgagcggaattgctacactaactaaggtgtgtttgtcctcatgtaagtagctcatcattaataaagatctggaaagatattgcaatgaatggaaatggttattgtcattgatgtgttggttccagtattccaaagttaagtcaagcgtactagatctgtgactggtcaattgttctatgtccgaaattactgaattttgttgcagaacatatgtgttgatccaaatttacacattcctgtatgaattatgatatataaaatcaatgaaatatccttcaacgtttcgagtacaatagtccagagtgcaagtcttggaaacagattccctgtactctttataaggctgtgtacgtccgctcttctgatacgttagatggtgtgagagccacacactgaaaacaattctgcaatcagtctatgacaaagattcctcagtttaatccagtgttgcgcggactctcaaaaatgatgccgcacccgtgtcggatcctccaaaaatacacttttttggaggatccgacacgcacccaataacattttcggagagtccgagaaacataggtttaatccatcacaaacaataggtgcaaatgcacatgcaaagacgtaactatacacatgccattttctagaactcagtagatgagaataaattaacgaatgaatgttaacttaaattataaagtaccaacaaaatttgtaactaataagaacggttcaaatattcacccggaaatgaaataatgtgtgaaatctaaacagaattgttgttggaatttgcaaaaggagctgtgagttttgatacttttggtacaactacttgcgtttacctttcaatggatcccctttggttctctaaatcaggtaacaaatatcaaatatttgtccacaaaaattaattccatgtgttcctttttcaggatatcgacactgctgctggtgtcatgttttaaaagaatatgaaaaatttcattgaaccaaaaacaaaatatcttcgtctctctaatgaaagacaatctagatgttttgtacctgataatgctatttcaagatgtcatggagttcattgtcttgcctctgtattaatgattagaatttagaagagagtgatttttttaatatttgcaggcgatggcagatgctgctcaccctgctaccatcttggagactaggaaaactgctccaggattacgtttggtggataaatgggcggtaaatatttactcgccaaaatcatccattgtgaaaggctatcacatgtatatatagtttatcttggctgactttctacctggctttaagtgaaggtattaatcggtggggggaagaatcacagaatgggcttatttgatatggtaatgataaaagataatcacatatctgctgctggaggtgtcagcaaagctctaaattccgtggatcagtatttggaacaaaataaacttcagatgggggttgaggttggccacatatacctttggattaaattgttcgatatggttatagtttgcgcatatgatatgtttgtttcgacctgacagttcaattacctattctgctggaatatattaatcatttaatcttagcttctgccatcagttgtgcaatactcttttgtcaattttgtattttgtcagataaacatttttctagagccatttcaatcataaactatttatattgttgataatataaactttatgattgatgctatttctgatatcctttcactgaagaactgaattcttatctgttgaaatcaaatgttagttcaagttcagagatgtttgccctacacctcaagacttttattttacatttttgtatctagatacactttaggggttgagagtagtatcccaaaatatatcatgatttcggataactgaaacaagagtggaaaattgttttatttgaccatgttaaagcagttagcgaacaaaatagtttgtcaggttacattttggactagttttcgaggtcatgttttttggatttcggcacacaacaatgtacatgggttccactcccttggatattctttcttgataatgttttgcttttgttacaaaagtaaagggaaaagtaaaaataacattatatattgctgaaaaaaccgggaaaatttctgctttcgggaatttttcaatgttatcatccaccctaatataacctaagaggtttgggagaaactgtatttttggtctgtctttgcataacacttaatagtgttgtcattaagttggtaatttgcacctactttggatagttaagaagactaattatgtagatactttttttttataaggcaaaatcttataaatgtgtcaccatcaagaatatgctggtaggaacccaacttacgttattgtatttagcagttttgagcgtatggtcctttctgaatgtgctcaatatctggtaattattgtcagagttttaaaccttttctcttatccaacaggttgaaaccaggacaattgcagaagtacatgaggttctagaatatgcatctcaaacaaagacttcgttgactaggataatgcttgataatatggttgttccattatctaacggagatattgaggtatccatgcttgaggaggctgtagatttgatcaatgggaggtttgagacagaggtacttctggtttcatttaaaatttttactgctccacaaactgtcagtatatacgaagcttagaaatagcatttaagatttgattttatgaaaaaaactcctaactcataaaaaaagatactgcgcggtttcagaggcgaatccaggatttaatgcttatggtttcctgcaacaatctcgagtaaattttcaataataactgagttcacattcaaacatttatagatatttagtaaattttcgaacacatttacactgtttggacaaaatctactagattcacttgaacccttaggtcgggaggtggatccgccccttcttggtttcccgtttattatttttgtcagcggatatcttgtgatgacatgctttgtttgctgtaatggtggcaggcttcaggaaatgttacccttgaaacagtgcacaagattggacaaactggtgttacctacatttctaggtatgtatttcgctcttgctattttttagacaagaggggttgctctgatgataagcaacctccactttcaaccaagaggttgtgagttcgagtcaccccaagagcaaggtggggagttcttggagggaaggatgtcgagggtctattggaaacagcctctctacctcatggtaggggtaaggtctgcgtacacactaccctccctagaccccactagtgggattatactgggttgttgttgttattttttcagaagatcttcattaaagaaattggaagataataaaaatagaatagacttctcattgtgtgttatttgtattgctgggacgtctttcttttcctctttttttatactaataagatggttagtttactcgcacacatcagacaaaagctcccattagatggtctatgttatgcataattttcttgataaaattgataaacaggagaacttacatggattgctaaagtacaacaacaactataaacctagtgtaatcccacaagtagggtatggggaaggtagtgtgtacgcagaccttactttcatcttgaaggtagagaggtggttttcgatagaccctcggctcacgaaagacgagaagaaaagcaatagcaccaagcaataaccaataagatagtaagagaacgaaacgaaaggaacaagtaatactaaaaaaatctaacaaaaatagaatataggactaatactctcgacgacctacttaccgtctaccttgattcttgagcttcacacattcctatcaagggtcatgtcctctgtaagttgtagtaaagtcatatcctgcctaatcacctctcctctatacttcttcggccaatctctacctcttctcagcccctttagcttcaacctctcacacctccttacatggattgctaaagtgctgacaattaggttgttgacctcttcggttatttataagttaacaaaggctttacttttaagcacatgatttagtgtggggtacaatttcctcgctaggagtggactcaatacttgagaaagttcaatatatgtttgtttcatccactaatgataccctttgtcagatattagcctgttattttgataaaaacgaatctatcttttgtttggtccatatgtattaggcacctgaaggttttgttatgatacttatagtatgaatatttgcagtggtgcactaacgcattctgtgaaagcacttgacatttccctgaagatcgatacagagctcgcccttgaagtcggacgacgtacaaaacgagcatgaccgccattacttttgttgtagggttggagtaaaagcagctgaatggcacaaagtgcaaataagaattattttactagttgtcaaacaaataagatgtaagttgctggaataattcggtggctcttttacaacctaattgcttgagttgatatttcattatagctttgctttcatgttttggtgtgtgtgaagttctgtgttacttcaaatcttttatggctctcttccaacctacctacctctgaatattttggtgcactactttgtaaaggaaacagcaaagt

SEQ ID NO: 2 - DNA, 6607 bp, Nicotiana tabacum
  source 1..6607: mol_type = genomic DNA
tttttgctatttttagtatatttttcttttgtctttagtacaattttttttcttcgctttttaggatttgatagacctttcctaaggttaaatctttttttatcataatactcatcaaatctaagagacacagagctcattaatagcccgtttggccaagctgcaaaaatcagcttattttgataagtgctttttttcaaaagtacttttgatgagaagcagtttgtgtttggctaattagtttaaaaagcacttctgagtagcaattagtgtttcgccaagctttaaaaaactgtttctaagtgtatttctcaaaagtgcttctcaaaaaagtaattttggagagaagctactttttttctgcttctccaaaattatttttttccttccagaagcttggccaaacacctcaatttttggccaaaagtacttttggcaaaaaaaaaaaagaacttttggccaaaaataagcttggccaaattttaggaccaaaagtattcagagtatacttaagggactactttgaacttatcctcaaacataagggactatatttgtcattttctcgaagttctcacaacccaagaaaccactaagctcagcaaagctatttgctccaaaaatcaaaatttcaatgtttaaagtttttcctttcactgcaatagtgcaccctcatgcaattacagcaccaaggtttctttagaacccctcaatttaacccagaagcaaaaaaaaaaaaaaggattcttgaaaatcatgtttttatttttgttatatatactgataaagattgttcttttatgcaattacagctcccaatgtttctttaaaaccactcaattccacccagaaaggaaaaaaaaagattcttgaaaatcatgttcttattgttttgtactgataaagattattcctttatgagtttctgattaaattttgtctgtaaattgcaggttggttgtgaaaatgtcagcaatagccaccaaaaatgcagtggagtcattagtagtgaagccaccagcacacccaacttatgatttaaagggtgttattcaacttgccctctctgaagatgctggggatttaggtttggttttagctcatattattttaatcttattatattgtcaaatgaggtgcactcagtttcaatactttagtaaatactgttaagggaagctaatggtagactttttaattaattgggagtagctttatgaagtttagctatagtctttttttgttttgtgtgaactccaagcttcaatttttcttgtgtagcctcaattaatttatagctaattgtgttctttttccataagaacagctaatgtgttcttaataaggattatcttcagtttaaccgggggggggggggggggaggagaatagttctgccaaaagttgttttcctttcttgtttcccacctggtgtccggtacccgctttgtggcccaactaatccggagaactcttgttaaggattgaggagtatgtatcgccccaccgcaacctttggtgtcaatagttgtttttttaagttcaagagcccgaggatattgtcatgtactgtctgactctctgcgtgatggaaaagtttgtttgagtaaatttcttggtactagcaatattttcagcacctagctaccattgtgaatttgagtactcaattataaaaaaccaatttgtcctgtgtattatttgtgtgaaatgcaggagatgtgacttgtaaggcaacaattcctattgacatggaatccgaagctcattttctagcaaaggaagacgggattgtagcaggaattgcacttgctgagatgatattcgcagaggttgatccttcactaaaggtattgattttttcaattaggttgttctggtaaccggtatttctactttgaagaaatgtctgaaaaaaggttttttaatttcttcatgttctcattatcagatggagtggtctataaatgatggtgataaagttcataaaggcttgaaattcggcaaagtacaaggtaaactatatgtgagaaatgatgaattactaatgattttttgaattaataataatcttgccttgacgtctgcaggaaaggctcacagcattgttatagctgagagagttgttctcaatttcatgcaaagaatgagcggaatagctacactaactaaggtgtgtttgtcctcatgtaagtagcacatcattaataaagatctggaaagatagcaatgaatgcaaatggttatgtcattgatgagttggtttcagtattccaaagttaagtcaagcgtaccagatctgagactgtcaattgttctatgcccgaaattactggattttgttgcagaacatatgcattgatctaaatttacacattcctgtatgatatataaaatcaatgaaatatccttcaacggttcaagtacaatggtcaagagttcaagtcttagaaacagattccgcgtattctttataaggctgtgtacgtccactcttctgatacattagatggtgtgagagccgcacactgaaaacaattctgcaatcagtctatgacaaagattcctcagtttattccatcacaaacaaaaggtgcaaatgcacatgcaaagacttaactatacacatgccattttctaaaactcagtagacgagaataaattaactaatgaatgttaacataaattatgaagtaccaacaaaatttgtaactaataagaacggtacaaatattcacccggaatgaaataatgtgtgaaatctaaacagatattgctgttagaatttgcaaacggagctgtgagttttgacacttttggtacaactacttgcgtttacctttcaatggaacccctttggttctctaaatcaggtaataaatatcaaatatctgtccacaaaaattaattccatgtgttcctttttcaggatatggacactgctgcaggtgtcatgttttaaaagaatttgaaagatttcattgaaccagaaccaaacatcttcatctctttaatgaatagacaaactagacattttgtacctgataatgctatctcaagatgtcatggagttcattgtcttgcctctgtagtaatgattagaatttagaagagagtgagttctttaatatttgcaggcgatggcagatgctgcacaccctgctaccatcttggagactaggaaaactgctccaggattacgtttggtggataaatgggcggtaaatatttactcgccaaaatcatccattgtgagaggctatcacatgtatatatagttcatcttggctgactttctacctggctttaagtgaaggtattgatcggtggggggaagaatcacagaatgggcttatttgatatggtaatgataaaagataatcacatatctgctgctggaggtgtcagcaaagctctaaattccgtggatcagtatttggaacaaaataaactccagatgggggttgaggttggccccatttacctttggattaaattgtgtggttatagttatgtttatttcaacctgacagttcaattacctattctgctggaatagattaatcatttaatcttagcttctgccatcagttgtgcaatactcttttgtcagttttgtatttggtcagataaatatctttctagagctattttcaatcaaaaactatttatagtgttgatgatataaactttataattgatgctatttctgatatcctttcattgaagaattgaattcttatctgttgaaatcaaatgttagtccaagttcagagatgtttgcactacacctcgagacttttattttacatttttgtatctagatacacttgtaggggttgagagcaacaacaacaaacccagtataatcccacaagtggggtctggtgagggtggtgtatacgcagaccttgaagctagagaggctgttttcgaaagacccccagcgtagaggttgagagtagtatcccagaatatatcatgatttttgataactgaaacaagagtggaaaattgtgttatttgaccatgttaaagcagttagcgaacaaaatagtttgtcaggttacattttggactagttttcaaggtcatcttttttggatttcggcacacaacaatgttcatggttccactcccttgggtattctttcttgataatgttttgcttttgttacaaaagtaaagggaaaagtaaaaataacattatatattgctgaaagagctgggaaaatttcttcttccaggaatttttcaatgttatcatccaccctaatataacctaagagccaaaaggtttgggggatattgtattttaggtctgtctttgcatactacttattggtgttgtctttcattaagttggtaatttgcacctacttttggatagttaagaatactaataatgtagatactttttttataaggtaagatcttataaatgtgtcagcatcaagaatatgctggtaggaacccaaattgtctgatttaagatacccgaacttacgttattgtatttagcagttttgagcatatggtcctttctgaatctgctcaatttctggtaattattggcagagttttaaaccttttctcctatccaacaggttgaaaccaggacaattgcagaagtacgagaggttctggactatgcatctcaaacaaagacttcgttgactaggataatgctggacaatatggttgttccattatctaatggagatattgatgtatccatgcttaaggaggctgtagatttgatcaatgggaggtttgagacagaggtacttctggtttcattttaaaattttactgctccacaaactgtcagtatatacgcagcttagaaatagcatttaagatttgattttatgataaaaactcctaactcataaaaaatggtactgcttggtttcccgtttattatttttgtcagtggatatcttgtgatgacatgctttgtttgctgtaatggtggcaggcttcaggaaatgttacccttgaaacagtgcacaagattggacaaactggtgttacctacatttctaggtatgtatttcgctcttgctattttttcagaagatcttcattaaaaaaattggaagataataaaaatagaagagacttcttggtttgtgttatttgtgttgctgcgacgtctttcttttcctctttttttttatactaataagatggttagtttactcgcacacatcagacaaaagctcccattagatagtctatgttatgtataattttcttgataaaattgataaacaggagaacttgcatggattgctaaagtacaacaacaacaacaacaacaaaatacccagtgtaatcccacaagtggagtctagggaggatagtgtgtacgcataccttacccccaccttgaaggtagagaggctgtttccgatagactctcggctcatgaaagacgagaagaaaagcaatagcaccaagcaataaccaacaagatagtaagagaacgaaacgaaaggaacaataagtaatactaaaaaaatctaacaaaaatagaatacgggactaacactaatactctcaactacctactagtcgtataccttgagtctcgacctccacaccttcctatcaaggatcatgtccttggtaagctgaactaatgtcatatcctgcctaatcacctctccccaatacttcttcggcctacctctacctcttctcaacccctccagctccaacctctcacacctccttacctggattgctaaagtacttaaaattattaggttgttgacctcttcggttatttattggttaacaaaggcttcacgtttaagcacatgatttagtgtggggtacaatttcctcggctaggagtggactcaatacttgagaaagttcaatatatgtttgtttcatccaggaatgatgccctttatcagatattagcctgttattttgataaaaaacgaatctatcttttgtttagtccatatgtattaggcacataaaggtttgttatgatactcatactatgaaaaatttaatgtttgcagtggtgccctaacgcattctgtgaaagcacttgacatatccctgaagatcgatacagagctcgcccttgaagttggacgacgtacaaaacgagcatgaccgccattacttttgttgtagggttggagtaaaagcagctgaatggcacaaagtgcaaataagaatcattttactagttgtcaaacaaataagatgtaagttgctggaatatttcgatggcttttttccaacctaattgcttgagttggtatttcattatatctttgctttcatgtttcatggaatttgacacaatgtaaaaagttgatttaaaagtttggtgtatgtgaagttttgtgttacttcacatcttttatggctctcttccaacctaaattcagtcaaacctctctctaacagcctcttttgttccgatattttttggttgctatcgtgaagtgctgttataaaagacatatattataatatagcataaaaatccgtttcgaaaaaaatttgacttttatagtgaattgttgttatataaggatgtatggagatatctgactgtagctctgtatatttt

SEQ ID NO: 3 - DNA, 5494 bp, Nicotiana tabacum
  source 1..5494: mol_type = genomic DNA
aaaatccaacggacaaaaaaatcggctgaatttgatttggttccaacatttaaaaaagtttcagtgagaaagaatacggtgactgttgatgatataaacaaagggcacattggtcaataaccataaaaaattatatgacagctacagttggtagcatgtgctcagctattgaacaaatctaaagaaggtacatctgtaaccggaacagcacttaaatgactaaattaccctcatcagaaagcagatggagtgctacaaataacacactattcaacaaccataaataaaacgtgttcagctactaaaacaaatataaataaatctatgtttgtaagcactccagccatgttaatggagtgctattgcctgttaactctcacttataaaatagtagtagaaaaaatatgaaccaaaacacaaccaaagaaagcattaagctccccaaaaactattttccacaaaattcatttcacaacccccccaaaaaaaaaccatgtttagagctattcctttcactgctacagtgcatccttatgcaattacagctccaaggtctctttaagaagaaaaaaagattcttgaaaatcatgtttttttggttatattctgataaagattgggttttttctgagtttgtgattaaattttgtgtgtaaaattgcaggttggtggtgaaaatgtcagcaatagccaccaagaatacaagagtggagtcattagaggtgaaaccaccagcacacccaacttatgatttaaaggaagttatgaaacttgcactctctgaagatgctgggaatttaggtttggtcttagcttatatcttttttctttttaatcttattatattgttaaatgggttgcactcaattttattactttagtaaatgctgtaattacctaatatgtggcctaactgtgtatatattttttacccgttagtgcatagaacttaagcaatttatttaattttggagttgctttatgaagtttagctatagttctttttgttagtaagtgttacctttagtttaaagaagggtgtgggtagaggcagatacacgatttgtgttcaacctttaaggttcttagtattgaatctatcatatttttgaagttacgggttcagattcaccatgtgttgcaattttagtgaatttttacactaaaattatgccccgtgtccaaactggtactataacgctacatatgcctctgttgggtggattgtggggggggggggggatagctctgtcaaaagttatttttttggtttctcgcccggtgtccgttgcctggtttgtggttcgaccaatccgtttgaggtaaaggaacgactccattctaatctgagacatatggttaaagatagagggtactaaccaccccacttacaacctttggtggtaaaagttacattatttaagatcaatagttgtggatattgtcatgtactctatcagtttgtgcttgatgggaaactttgtgtgattaagtttcttggaaatagcaattttttaagcacctggttacctttgtgaatttgagtactcaatttgaaaaagagatgtctatttattcaatgttttcttgggtgaaatgcaggagatgtgacttgtaaggcgacaattcctcttgatatggaatccgatgctcattttctagcaaaggaagacgggatcatagcaggaattgcacttgctgagatgatattcgcggaagttgatccttcattaaaggtattgattttgctattaagttgttccagtaaccggcatatctatttcgaaaaagtttttgataaagggtaatttttaatttcttcatgatcttattatcaggtggagtggtatgtaaatgatggcgataaagttcataaaggcttgaaatttggcaaagtacaaggtaaactaaatgtgataaaatgatgacttgctaatgatttttcagcttattaatctttccttgacttttgtaggaaacgcttacaacattgttatagctgagagggttgttctcaattttatgcaaagaatgagtggaatagctacactaactaaggtgtgtttctcttcatataaatagtttatccttaaaaaaaatatggaaagatattgccatgaatggaaatggttagtgtcattgatgagttggtttcagtatttcaagttaagtgtaggtctgagactgctgaattgttttatgtcgaagaatactgagtttcgtgcagaacatatgtgttgctccatatttagacattcctctatgatataaaatagctattgttgttcgtcaggttgtgcaattggcttgttaggtcattgatccagctcaaagactgttgaactaagagactttctccgtatttgcatttcaatgtattatggttgaaaaactaaagtctccattcttcaatatttcgagtatgatagcttctctgagatgactatttgccactgatgttacttgcttggcgtgtgagtgctattagtatgaccttatttggattatttttttcttcttcttaataacggtggttcccgtttagtttgcgtgcgcctcgactattccactgggtacatgctgccttccatcggcacaggtatcaggtaactctgttaatgaaggcttagggataaaagggcagcccggtgcactaagctctcgctgtgcatggggtccgaggaagggccggaccacaagcttttgttgtcttttggaatttgaatcctggtatcccatagtttacacccacttcattgaccgctaggttgtcccttgggtgctaatgttatttatattttgaaacttcttgtttgtcttttatgctgaagatggtcctgaataggctagcattgtggggatccaacttcctgaattcattgttttcatatttctatgagaagtagccttgaccagaaagataagagttcaagtcttggaaacaaatcgtgtgtattcgttataaggctatgcacatccactcttctgagaccttagatggtgccagagtaatatattgaaaactattttgcaatcagtctatgagaaagactcctctgtttaactaaataattccattccgagatgtcaattagcttaatttcttgcctctatagtagtgattagaagggactgagtttttttttttatttgcaggaaatggcagatgctgcacaccctgcttacatcttggagactaggaaaactgctcctggattacgtttggtggataaatgggcggtaaatatttactcaccaaaatcatctacttgtcaaggctatcacatgtatagtttatcttggctgactttctacctggctttaattaaaggtattgatcggtggggggaagaatcacagaatgggcttatttgatatggtaatgataaaagacaatcacatatctgctgctggaggtgtcggcaaagctctaaaatctgtggatcagtatttggagcaaaataaacttcaaataggggttgaggttggcctcatatacctttgaattaaattcttcgatatggttttagatttgtgtccttatcttttaattttatcgtttgcacatttgatatgtttatttggacctgacagttcaattacctattctaccatccgttacgctatacttttttgtcagttttggtcagataaacatctttctagagccattccaatcataaactatttatatttttgaagatataaactttatggctgatgctacttccaatatcctttcactgaagaactaaattattttctgttgaaaacaaatgttagtccaagaaagagatgtttgacctatatgtgtctttcattaagttggtattcgctcctactttggatagttaagaagactatagataaaatttgacaaggtaaaatttaataaatgtgtcagcatcaagaatatgctggtaggaacccaaattgtctgatttgcgatccacgaagttacgcaattgtatttggcagtagagctgtggtcctttcataatgtattctatttgtggtattgaacagagttttaaaccttttctcctatccaacaggttgaaaccaggacaattgaagaagtacgtgaggttctagactatgcatctcaaacaaagacttcgttgactaggataatgctggacaatatggttgttccattatctaacggagatattgatgtatccatgcttaaggaggctgtagaattgatcaatgggaggtttgatacggaggtaacgcctggtttcattttcattaactatttcacacgtgtcaactatgttgctcggactcttcaaatatgtagcaggatgcgtgtcaaatcctccaaaagtagtgcatttttggaggatctgacaggggtgcggcatcattttaagagaatctgcgcaacatagagtgtcagaatatggaagacttactgagatgcaatgccttgatttttcataaaaatgaagtaacgcctggatttcctgtaatcgtatatcttttggtgacatgctgttgttgttgtaatggtggcaggcttcaggaaatgttacccttgaaacagtacacaagattggacaaactggtgttacctacatttctaggtatgcatttcgctcttgctatttttttccaaagatatccatacaagacagcagaagacacatatataagagatttctcagtgtttgttattagtatagctgggatgcctttctgtttcttttcttttccttcttataataagatggttagtatacttgccttgttcagacaaacgaaaaagatggttactttacttgcgcatgttagaccaaaaatggtctcagtagatagtctatgttatgtaacaattcttttgataggcaaaagtgacaggtttttatccttagtattaggtgcgcgaaggtttgttatgatactaaaaatagagtttaaatctttgcagtggtgccctgacgcattccgtgaaagcacttgacatttccctgaagatcgatacagagctcgcccttgaagttggaaggcgtacaaaacgagcatgagcgccattacttctgctatagggttggagtaaaagcagctgaatagctgaaaggtgcaaataagaatcattttactagttgtcaaacaaaagatccttcactgtgtaatcaaacaaaaagatgtaaattgctggaatatctcagatggctcttttccaaccttattgcttgagttggtaatttcattatagctttgttttcatgtttcatggaatttgttacaatgaaaatacttgatttataagtttggtgtatgtaaaattctgtgttacttcaaatattttgagatgttgaatatcatgttcttaagctggatcagcaggatatagtcactaaataatttctgtgtctgctcatttacaaagatcaatttatccagctcattcttagggagtagtaataaactgaagaattaacccaaatagccgcacacccaacatttaaactaaaaatagccggtg

SEQ ID NO: 4 - DNA, 4762 bp, Nicotiana tabacum
  source 1..4762: mol_type = genomic DNA
tatggccaaacggccccttaattgatttatttttcttttaaggtgactattgattatataagcaaagggcacattggtcaatagccataaaaatttatgtgacagctgtagttggggagcatgtgctcagttattaaacaaacaaatctaaagaaccctgtacacatgtaatcagaacagcacttaaatgactaaattaccctcatcagcaaagcagatggagtgctacaaataatacattggtcaacaaccaaaaataaaacgtgttcagctactaaaacaaatataaagaaatctgtgtttgtaagcactccagccatgttaatggagtgctattgacagttaactcacttataaaatagtagtagaaaaaatatggaccaaaaacacaaccaaagaaagcattaagctcctcaaaaactattttccacaaaaatcatttcacagccccaagaaaataaaccatgtttagggctcttcctttcactgcaacagtgcatccatatgcaattacagctccaaggtctctttaagaagaaaaaaagattcttgaaaatcatgtttttttggttatattctgataaagattggatttttatgagtttctgattgaattttgtgtgtaaaattgcaggttggtggtgaaaatgtcagcaatagccaccaagaatacaagagtggagtcattagaggtgaagccaccagcacacccaacttatgatttaaagggtgttatgcaacttgcactctctgaagatgctgggaatttaggtctggtcttagcctatattttttattttatcttattatattgtcaaatgggttgcactcaattttattattttagtaaatgatgtaattacctaatatgtggcctaattgtgtatatatatattttacactgttagtgcatagtaacatataacttaagctctttatttaatttgtagttgctttatgaagtttagctatagtattttttttttggggggcgggggagctccaagcttcaatttatttttaattgtgtagcctcatttacttacagataatgtgttcttagtaagtgttacccttagtttaaggaagggggttgggggtaggggcggatccaggagggtattgggggtagaggcaggagggtattgggggtagaggcggatccgcaaatacccttagttatttttattggtttctcgccccatgtccggtaattgctttgtggtccgactaatccggattgaggtaaaggccgacttcattctcaggcttgaacctgagacatttggttaaggatggagggtactaaccaccccacttacaacctttggtggtaaaagtcgcattttttaagatccaagagttgaggatgttgtcatgtactctatcagtttgtgcttgatgggaaactttgcttaattaagtttcttggaaatagcaaatttttaagcacctagttacctttgtgaatttgagtactcaatttgaaaaagatatatctatttattcaatgtttccttgtgtggaatgcaggagatgtgacttgtaaggcgacgattcctgttgatatggaatccgatgctcattttctagcaaaggaagacgggatcatagcagggattgcacttgctgagatgatattcgcggaagttgatccttcactaaaggtattgattttgcaattaagttgttccagtaaccggcatatctatttcgaagaagttttttatacagggtaatttttaaattgttcatgttcttattatcaggtggagtggtatgtaaatgatggtgataaagttcataaaggcttgaaatttggcaaagtacaaggtaaactaaatgtgataaaatgatgaattgctaatgatttttcggcttattaatctttccttgacttttgtaggaaacgcttacaacattgttatagctgagagggttgttctcaattttatgcaaagaatgagtggaatagctacactaactaaggtgtgtttctcttcatataaatagctcatcactaaaaaaaatatggaaagatattgccatgaatggaaatggttagtgtcattgacgagttggtttcagtatttcaagttaaatgtaggtcagagactgctgaattgttctatgtcgaagaatagaacaatcaaatgatatataatcaataaaatatgttggtcatgaatgcggtaatgtatgacttaaaatcttgtgatggtacttctagtttaaccaatattcttactgatcaaatacttgaaccaatactcaagcttcattatcaagctcatagactgttgaagtaggagactttgtctgaatttgcaattcaatgagaattagccttaacctatggtaaaagttctccatctcaccagaaagtcaagagtgcaagtcttggaaacaaatcctgtgtaatcgttataaggctgtgcacatccactcttctgagactttagatggtgccagagccacatattgaaaaccattttgcaatcaaacaacaacatacctagtaaaccaacaatgaggtctggggagggtagcaagtacgcaggccttacccctaccctgtgaaggtagagagactgttttcgatagaccctattttgctctcagtctatgagaaaaactcctctgtttaactagataattccattccgagatgtcaatgagtttaaattcttgcctctgtagtagtgattagaagagagtgagactgggtttttcttttatttgcaggaaatggcagatgctgcacaccctgcttacatcttggagactaggaaaactgctcctggattacgtttggtggataaatgggcggtaaagatttactcaccaaaatcatttacttgtcaagtccatcacatgtatagtttatcttggctgactttcaacctggctttaattaaaggtattgatcggtggtgggaagaatcacagaatgggcttatttgatatggtaatgataaaagacaatcacatatctgctgctggaggtgtcggcaaagctctaaaatctgtggatcagtatttggagcaaaataaacttcaaataggggttgaggttggcctcataccttttaattaaattctttgatatgtttataaatctgagtctttatcttttagttttattgtttgcacctttgatatgtttacttggacctgacagttcaattacctattctaccaaccgttatgctttactttttttgtcatttttggtcaaacatctttctagatatgtttgacctaaatatttctgtctttcattaagttggtatttgcacctactttggatagttaagaataattatgtagatacttttttttgataagtaaggtaaaattttataaatatgtcagcatcaagaatatgctggtaggaacccaaattgtctgatttgtgatttccgaagttacgatattgtatttggcagtagagatgtggtcctttcagagtgtgctcaattttgtggtaattattgaacagaggtttaaacgtttttctcctatccaacaggttgaaaccaggacaattgcagaagtacgtgaggttctagaatatgcatctcaaacaaagacttcgttgactaggataatgctggacaatatggttgttccattatctaacggagatattgatgtatccatgcttaaggaggctgtagaattgatcaatgggaggtttgatacggaggtaacgcctggtttcatttgcattgtctgcttcacataagtgtcagaatatagaagaactgtgatgcaatgccttgatttttcataaaaaagaagtaccgcatggatttcctgtaaacgtatatcttttggtgacatgctgttgttgttgtaatggtggggcaggcttcaggaaatgttacccttgaaacagtacacaagattggacaaactggtgttacctacatttctaggtatgcatttcgctcttgctaatttacttcaaaagatatccatagaatacagcagaagacatatagaagagatttctcagtgtttgttactattagtatagctgggacgtctttctgttccttttgttttccttcatataataagatggttagtttaacttgccttgttcagaaaaacgaaaaagatgattagtttacttgcacacaatagaccagaaacggtctcagtagatagtctatgttatgttaataattcatttgataggcaaaagtgacaggtttttatccttagtattaggcgcgcgaaggtttgttatgatactaaaaatatagagtttaaatctttgcagtggtgccctgacgcattctgtgaaagcacttgacatttccctgaagattgatacagagctcgcccttgaagttggaaggcgtacaaaacgagcatgagcgccattacttctgctatagggttggagtaaaagcagctgaatcgctgaaagttcaaataagaatcattttactaatgtcaaacaaaaaagatcgatctttcactgtgtaatcaaacaaaaagatgtaaattgctggaatatctcagatggctcttttccaaccttattgcttgaattggtatttcaatatagctttgttttcatgtttcatggaatttgttacaatgaaaatacttgatttacaagtttggtgtatctgaagctctgtgttacttcaaatcttttgagatgttgaatatgttcttaagctggatcagcaggatatagtcaccaagtaatctgtctaacatacaaagatcaatttgtccactt

SEQ ID NO: 5 - DNA, 1053 bp, Nicotiana tabacum
  source 1..1053: mol_type = genomic DNA
atgtttaaagttcttcctttcactgcaatagtccaccctaatgcaattacagctccaagattggttgtgaaaatgtcagcaatagccaccaaaaatgcagtggagtcatttgtagtgaagccaccagcacacccaacttatgatttaaagggtgttattcaacttgccctctctgaagatgctggggatataggagatgtgacttgtaaggcgacaattcctattgacatggaatccgaagctcattttctagcaaaggaagacgggattgtagcaggaattgcacttgctgagatgatattcgcagaggttgatccttcactaaagatggagtggtctataaatgatggcgataaagttcataaaggcttgaaattcggcaaagtacaaggaaaggctcacagcattgttatagctgagagagttgttctcaattttatgcaaaggatgagcggaattgctacactaactaaggcgatggcagatgctgctcaccctgctaccatcttggagactaggaaaactgctccaggattacgtttggtggataaatgggcggtattaatcggtggggggaagaatcacagaatgggcttatttgatatggtaatgataaaagataatcacatatctgctgctggaggtgtcagcaaagctctaaattccgtggatcagtatttggaacaaaataaacttcagatgggggttgaggttgaaaccaggacaattgcagaagtacatgaggttctagaatatgcatctcaaacaaagacttcgttgactaggataatgcttgataatatggttgttccattatctaacggagatattgaggtatccatgcttgaggaggctgtagatttgatcaatgggaggtttgagacagaggcttcaggaaatgttacccttgaaacagtgcacaagattggacaaactggtgttacctacatttctagtggtgcactaacgcattctgtgaaagcacttgacatttccctgaagatcgatacagagctcgcccttgaagtcggacgacgtacaaaacgagcatga

SEQ ID NO: 6 - DNA, 1053 bp, Nicotiana tabacum
  source 1..1053: mol_type = genomic DNA
atgtttaaagtttttcctttcactgcaatagtgcaccctcatgcaattacagcaccaaggttggttgtgaaaatgtcagcaatagccaccaaaaatgcagtggagtcattagtagtgaagccaccagcacacccaacttatgatttaaagggtgttattcaacttgccctctctgaagatgctggggatttaggagatgtgacttgtaaggcaacaattcctattgacatggaatccgaagctcattttctagcaaaggaagacgggattgtagcaggaattgcacttgctgagatgatattcgcagaggttgatccttcactaaagatggagtggtctataaatgatggtgataaagttcataaaggcttgaaattcggcaaagtacaaggaaaggctcacagcattgttatagctgagagagttgttctcaatttcatgcaaagaatgagcggaatagctacactaactaaggcgatggcagatgctgcacaccctgctaccatcttggagactaggaaaactgctccaggattacgtttggtggataaatgggcggtattgatcggtggggggaagaatcacagaatgggcttatttgatatggtaatgataaaagataatcacatatctgctgctggaggtgtcagcaaagctctaaattccgtggatcagtatttggaacaaaataaactccagatgggggttgaggttgaaaccaggacaattgcagaagtacgagaggttctggactatgcatctcaaacaaagacttcgttgactaggataatgctggacaatatggttgttccattatctaatggagatattgatgtatccatgcttaaggaggctgtagatttgatcaatgggaggtttgagacagaggcttcaggaaatgttacccttgaaacagtgcacaagattggacaaactggtgttacctacatttctagtggtgccctaacgcattctgtgaaagcacttgacatatccctgaagatcgatacagagctcgcccttgaagttggacgacgtacaaaacgagcatga

SEQ ID NO: 7 - DNA, 754 bp, Nicotiana tabacum
  source 1..754: mol_type = genomic DNA
atgtttagagctattcctttcactgctacagtgcatccttatgcaattacagctccaaggttggtggtgaaaatgtcagcaatagccaccaagaatacaagagtggagtcattagaggtgaaaccaccagcacacccaacttatgatttaaaggaagttatgaaacttgcactctctgaagatgctgggaatttaggagatgtgacttgtaaggcgacaattcctcttgatatggaatccgatgctcattttctagcaaaggaagacgggatcatagcaggaattgcacttgctgagatgatattcgcggaagttgatccttcattaaaggtggagtggtatgtaaatgatggcgataaagttcataaaggcttgaaatttggcaaagtacaaggaaacgcttacaacattgttatagctgagagggttgttctcaattttatgcaaagaatgagtggaatagctacactaactaaggaaatggcagatgctgcacaccctgcttacatcttggagactaggaaaactgctcctggattacgtttggtggataaatgggcggtattgatcggtggggggaagaatcacagaatgggcttatttgatatggtaatgataaaagacaatcacatatctgctgctggaggtgtcggcaaagctctaaaatctgtggatcagtatttggagcaaaataaacttcaaataggggttgaggttgaaaccaggacaattgaagaagtacgtgaggttctag

SEQ ID NO: 8 - DNA, 754 bp, Nicotiana tabacum
  source 1..754: mol_type = genomic DNA
atgtttagggctcttcctttcactgcaacagtgcatccatatgcaattacagctccaaggttggtggtgaaaatgtcagcaatagccaccaagaatacaagagtggagtcattagaggtgaagccaccagcacacccaacttatgatttaaagggtgttatgcaacttgcactctctgaagatgctgggaatttaggagatgtgacttgtaaggcgacgattcctgttgatatggaatccgatgctcattttctagcaaaggaagacgggatcatagcagggattgcacttgctgagatgatattcgcggaagttgatccttcactaaaggtggagtggtatgtaaatgatggtgataaagttcataaaggcttgaaatttggcaaagtacaaggaaacgcttacaacattgttatagctgagagggttgttctcaattttatgcaaagaatgagtggaatagctacactaactaaggaaatggcagatgctgcacaccctgcttacatcttggagactaggaaaactgctcctggattacgtttggtggataaatgggcggtattgatcggtggtgggaagaatcacagaatgggcttatttgatatggtaatgataaaagacaatcacatatctgctgctggaggtgtcggcaaagctctaaaatctgtggatcagtatttggagcaaaataaacttcaaataggggttgaggttgaaaccaggacaattgcagaagtacgtgaggttctag

SEQ ID NO: 9 - protein, 350 aa, Nicotiana tabacum
MFKVLPFTAIVHPNAITAPRLVVKMSAIATKNAVESFVVKPPAHPTYDLKGVIQLALSEDAGDIGDVTCKATIPIDMESEAHFLAKEDGIVAGIALAEMIFAEVDPSLKMEWSINDGDKVHKGLKFGKVQGKAHSIVIAERVVLNFMQRMSGIATLTKAMADAAHPATILETRKTAPGLRLVDKWAVLIGGGKNHRMGLFDMVMIKDNHISAAGGVSKALNSVDQYLEQNKLQMGVEVETRTIAEVHEVLEYASQTKTSLTRIMLDNMVVPLSNGDIEVSMLEEAVDLINGRFETEASGNVTLETVHKIGQTGVTYISSGALTHSVKALDISLKIDTELALEVGRRTKRA

SEQ ID NO: 10 - protein, 350 aa, Nicotiana tabacum
MFKVFPFTAIVHPHAITAPRLVVKMSAIATKNAVESLVVKPPAHPTYDLKGVIQLALSEDAGDLGDVTCKATIPIDMESEAHFLAKEDGIVAGIALAEMIFAEVDPSLKMEWSINDGDKVHKGLKFGKVQGKAHSIVIAERVVLNFMQRMSGIATLTKAMADAAHPATILETRKTAPGLRLVDKWAVLIGGGKNHRMGLFDMVMIKDNHISAAGGVSKALNSVDQYLEQNKLQMGVEVETRTIAEVREVLDYASQTKTSLTRIMLDNMVVPLSNGDIDVSMLKEAVDLINGRFETEASGNVTLETVHKIGQTGVTYISSGALTHSVKALDISLKIDTELALEVGRRTKRA

SEQ ID NO: 11 - protein, 351 aa, Nicotiana tabacum
MFRAIPFTATVHPYAITAPRLVVKMSAIATKNTRVESLEVKPPAHPTYDLKEVMKLALSEDAGNLGDVTCKATIPLDMESDAHFLAKEDGIIAGIALAEMIFAEVDPSLKVEWYVNDGDKVHKGLKFGKVQGNAYNIVIAERVVLNFMQRMSGIATLTKEMADAAHPAYILETRKTAPGLRLVDKWAVLIGGGKNHRMGLFDMVMIKDNHISAAGGVGKALKSVDQYLEQNKLQIGVEVETRTIEEVREVLDYASQTKTSLTRIMLDNMVVPLSNGDIDVSMLKEAVELINGRFDTEASGNVTLETVHKIGQTGVTYISSGALTHSVKALDISLKIDTELALEVGRRTKRA

SEQ ID NO: 12 - protein, 351 aa, Nicotiana tabacum
MFRALPFTATVHPYAITAPRLVVKMSAIATKNTRVESLEVKPPAHPTYDLKGVMQLALSEDAGNLGDVTCKATIPVDMESDAHFLAKEDGIIAGIALAEMIFAEVDPSLKVEWYVNDGDKVHKGLKFGKVQGNAYNIVIAERVVLNFMQRMSGIATLTKEMADAAHPAYILETRKTAPGLRLVDKWAVLIGGGKNHRMGLFDMVMIKDNHISAAGGVGKALKSVDQYLEQNKLQIGVEVETRTIAEVREVLEYASQTKTSLTRIMLDNMVVPLSNGDIDVSMLKEAVELINGRFDTEASGNVTLETVHKIGQTGVTYISSGALTHSVKALDISLKIDTELALEVGRRTKRA

SEQ ID NO: 13 - DNA, 24 bp, synthetic construct
  misc_feature 1..24: note = Description of Artificial Sequence: Synthetic oligonucleotide
  source 1..24: mol_type = other DNA
tagcaaaggaagacgggatcatag

SEQ ID NO: 14 - DNA, 11 bp, Nicotiana tabacum
  source 1..11: mol_type = genomic DNA
acgggatcata

SEQ ID NO: 15 - DNA, 20 bp, Nicotiana tabacum
  source 1..20: mol_type = genomic DNA
ggatcatagcagggattgca

SEQ ID NO: 16 - DNA, 20 bp, Nicotiana tabacum
  source 1..20: mol_type = genomic DNA
ggatcatagcagggattgca

SEQ ID NO: 17 - DNA, 11 bp, Nicotiana tabacum
  source 1..11: mol_type = genomic DNA
acgggatcata

SEQ ID NO: 18 - DNA, 11 bp, Nicotiana tabacum
  source 1..11: mol_type = genomic DNA
agacgggatca

SEQ ID NO: 19 - DNA, 10 bp, Nicotiana tabacum
  source 1..10: mol_type = genomic DNA
acgggatcat

SEQ ID NO: 20 - DNA, 743 bp, Nicotiana tabacum
  source 1..743: mol_type = genomic DNA
atgtttagagctattcctttcactgctacagtgcatccttatgcaattacagctccaaggttggtggtgaaaatgtcagcaatagccaccaagaatacaagagtggagtcattagaggtgaaaccaccagcacacccaacttatgatttaaaggaagttatgaaacttgcactctctgaagatgctgggaatttaggagatgtgacttgtaaggcgacaattcctcttgatatggaatccgatgctcattttctagcaaaggatagcaggaattgcacttgctgagatgatattcgcggaagttgatccttcattaaaggtggagtggtatgtaaatgatggcgataaagttcataaaggcttgaaatttggcaaagtacaaggaaacgcttacaacattgttatagctgagagggttgttctcaattttatgcaaagaatgagtggaatagctacactaactaaggaaatggcagatgctgcacaccctgcttacatcttggagactaggaaaactgctcctggattacgtttggtggataaatgggcggtattgatcggtggggggaagaatcacagaatgggcttatttgatatggtaatgataaaagacaatcacatatctgctgctggaggtgtcggcaaagctctaaaatctgtggatcagtatttggagcaaaataaacttcaaataggggttgaggttgaaaccaggacaattgaagaagtacgtgaggttctag

SEQ ID NO: 21 - DNA, 747 bp, Nicotiana tabacum
  source 1..747: mol_type = genomic DNA
atgtttagagctattcctttcactgctacagtgcatccttatgcaattacagctccaaggttggtggtgaaaatgtcagcaatagccaccaagaatacaagagtggagtcattagaggtgaaaccaccagcacacccaacttatgatttaaaggaagttatgaaacttgcactctctgaagatgctgggaatttaggagatgtgacttgtaaggcgacaattcctcttgatatggaatccgatgctcattttctagcaaaggaagactagcaggaattgcacttgctgagatgatattcgcggaagttgatccttcattaaaggtggagtggtatgtaaatgatggcgataaagttcataaaggcttgaaatttggcaaagtacaaggaaacgcttacaacattgttatagctgagagggttgttctcaattttatgcaaagaatgagtggaatagctacactaactaaggaaatggcagatgctgcacaccctgcttacatcttggagactaggaaaactgctcctggattacgtttggtggataaatgggcggtattgatcggtggggggaagaatcacagaatgggcttatttgatatggtaatgataaaagacaatcacatatctgctgctggaggtgtcggcaaagctctaaaatctgtggatcagtatttggagcaaaataaacttcaaataggggttgaggttgaaaccaggacaattgaagaagtacgtgaggttctag

SEQ ID NO: 22 - DNA, 746 bp, Nicotiana tabacum
  source 1..746: mol_type = genomic DNA
atgtttagggctcttcctttcactgcaacagtgcatccatatgcaattacagctccaaggttggtggtgaaaatgtcagcaatagccaccaagaatacaagagtggagtcattagaggtgaagccaccagcacacccaacttatgatttaaagggtgttatgcaacttgcactctctgaagatgctgggaatttaggagatgtgacttgtaaggcgacgattcctgttgatatggaatccgatgctcattttctagcaaaggaagacggcagggattgcacttgctgagatgatattcgcggaagttgatccttcactaaaggtggagtggtatgtaaatgatggtgataaagttcataaaggcttgaaatttggcaaagtacaaggaaacgcttacaacattgttatagctgagagggttgttctcaattttatgcaaagaatgagtggaatagctacactaactaaggaaatggcagatgctgcacaccctgcttacatcttggagactaggaaaactgctcctggattacgtttggtggataaatgggcggtattgatcggtggtgggaagaatcacagaatgggcttatttgatatggtaatgataaaagacaatcacatatctgctgctggaggtgtcggcaaagctctaaaatctgtggatcagtatttggagcaaaataaacttcaaataggggttgaggttgaaaccaggacaattgcagaagtacgtgaggttctag